(19) United States
(12) Patent Application Publication
(10) Pub. No.: US 2023/0312482 A1
(43) Pub. Date: Oct. 5, 2023

(54) SOS1 INHIBITORS

(71) Applicant: Mirati Therapeutics, Inc., San Diego, CA (US)

(72) Inventors: Matthew Arnold Marx, San Diego, CA (US); John Michael Ketcham, San Diego, CA (US); Christopher Ronald Smith, San Diego, CA (US); John David Lawson, San Diego, CA (US); Anthony Ivetac, San Diego, CA (US)

(21) Appl. No.: 18/018,422

(22) PCT Filed: Jul. 27, 2021
(86) PCT No.: PCT/US2021/043309

§ 371 Date: Jan. 27, 2023

Related U.S. Application Data
(60) Provisional application No. 63/057,563, filed on Jul. 28, 2020.

(51) Int. Cl. C07D 237/34; C07D 409/12

(52) U.S. Cl. CPC C07D 237/34; C07D 409/12

(57) ABSTRACT

The present invention relates to compounds that inhibit Son of sevenless homolog 1 (SOS1) activity. In particular, the present invention relates to compounds, pharmaceutical compositions and methods of use, such as methods of treating cancer using the compounds and pharmaceutical compositions of the present invention.

### FIELD OF THE INVENTION

The present invention relates to compounds that inhibit Son of sevenless homolog 1 (SOS1) GTP-mediated nucleotide exchange. In particular, the present invention relates to compounds, pharmaceutical compositions comprising the compounds and methods for use therefor.

### BACKGROUND OF THE INVENTION

The Ras family comprises v-Ki-ras2 Kirsten rat sarcoma viral oncogene homolog (KRAS), neuroblastoma RAS viral oncogene homolog (NRAS), and Harvey murine sarcoma virus oncogene (HRAS) and critically regulates cellular division, growth and function in normal and altered states including cancer (see e.g., Simanshu et al. Cell, 2017. 170(1): p. 17-33; Matikas et al., Crit Rev Oncol Hematol, 2017. 110: p. 1-12). RAS proteins are activated by upstream signals, including receptor tyrosine kinases (RTKs), and transduce signals to several downstream signaling pathways such as the mitogen-activated protein kinase (MAPK)/extracellular signal-regulated kinases (ERK) pathway. Hyperactivation of RAS signaling is frequently observed in cancer as a result of mutations or alterations in RAS genes or other genes in the RAS pathway. The identification of strategies to inhibit RAS and RAS signaling are predicted to be useful for the treatment of cancer and RAS-regulated disease states.

RAS proteins are guanosine triphosphatases (GTPases) that cycle between an inactive, guanosine diphosphate (GDP)-bound state and an active guanosine triphosphate (GTP)-bound state. Son of sevenless homolog 1 (SOS1) is a guanine nucleotide exchange factor (GEF) that mediates the exchange of GDP for GTP, thereby activating RAS proteins. RAS proteins hydrolyze GTP to GDP through their intrinsic GTPase activity which is greatly enhanced by GTPase-activating proteins (GAPs). This regulation through GAPs and GEFs is the mechanism whereby activation and deactivation are tightly regulated under normal conditions. Mutations at several residues in all three RAS proteins are frequently observed in cancer and result in RAS remaining predominantly in the activated state (Sanchez-Vega et al., Cell, 2018. 173: p. 321-337 Li et al., Nature Reviews Cancer, 2018. 18: p. 767-777). Mutations at codon 12 and 13 are the most frequently mutated RAS residues and prevent GAP-stimulated GTP hydrolysis. Recent biochemical analyses however, demonstrated these mutated proteins still require nucleotide cycling for activation based on their intrinsic GTPase activity and/or partial sensitivity to extrinsic GTPases. As such, mutant RAS proteins are sensitive to inhibition of upstream factors such as SOS1 or SHP2, another upstream signaling molecule required for RAS activation (Hillig, 2019; Patricelli, 2016; Lito, 2016; Nichols, 2018).

The three main RAS-GEF families that have been identified in mammalian cells are SOS, RAS-GRF and RAS-GRP (Rojas, 2011). RAS-GRF and RAS-GRP are expressed in the cells of the central nervous system and hematopoietic cells, respectively, while the SOS family is ubiquitously expressed and is responsible for transducing RTK signaling. The SOS family comprises SOS1 and SOS2 and these proteins share approximately 70% sequence identity. SOS1 appears to be much more active than SOS2 due to the rapid degradation of SOS2. The mouse SOS2 knockout is viable whereas the SOS1 knockout is embryonic lethal. A tamoxifen-inducible SOS1 knockout mouse model was used to interrogate the role of SOS1 and SOS2 in adult mice and demonstrated the SOS1 knockout was viable but the SOS½ double knockout was not viable (Baltanas, 2013) suggesting functional redundancy and that selective inhibition of SOS1 may have a sufficient therapeutic index for the treatment of SOS1 - RAS activated diseases.

SOS proteins are recruited to phosphorylated RTKs through an interaction with growth factor receptor bound protein 2 (GRB2). Recruitment to the plasma membrane places in SOS in close proximity to RAS and enables SOS-mediated RAS activation. SOS proteins bind to RAS through a catalytic binding site that promotes nucleotide exchange as well as through an allosteric site that binds GTP-bound RAS-family proteins which increases the catalytic function of SOS (Freedman et al., Proc. Natl. Acad. Sci, USA 2006. 103(45): p. 16692-97). Binding to the allosteric site relieves steric occlusion of the catalytic site and is therefore required for full activation of the catalytic site. Retention of the active conformation at the catalytic site following interaction with the allosteric site is maintained in isolation due to strengthened interactions of key domains in the activated state. SOS1 mutations are found in Noonan syndrome and several cancers including lung adenocarcinoma, embryonal rhabdomyosarcoma, Sertoli cell testis tumor and granular cell tumors of the skin (see e.g., Denayer, E., et al, Genes Chromosomes Cancer, 2010. 49(3): p. 242-52).

GTPase-activating proteins (GAPs) are proteins that stimulate the low intrinsic GTPase activity of RAS family members and therefore converts active GTP-bound RAS proteins into inactive, GDP-bound RAS proteins (e.g., see Simanshu, D.K., Cell, 2017, Ras Proteins and their Regulators in Human Disease). While activating alterations in the GEF SOS1 occur in cancers, inactivating mutations and alterations in the GAPs neurofibromin 1 (NF-1) or neurofibromin 2 (NF-2) also occur creating a state where SOS1 activity is unopposed and activity downstream of the pathway through RAS proteins is elevated.

Thus, the compounds of the present invention that block the interaction between SOS 1 and Ras-family members prevent the recycling of KRas in to the active GTP-bound form and, therefore, may provide therapeutic benefit for a wide range of cancers, particularly Ras family member-associated cancers. The compounds of the present invention offer potential therapeutic benefit as inhibitors of SOS 1-KRas interaction that may be useful for negatively modulating the activity of KRas through blocking SOS 1-KRas interaction in a cell for treating various forms cancer, including Ras-associated cancer, SOS 1-associated cancer and NF1/NF2-associated cancer.

### SUMMARY OF THE INVENTION

There is a need to develop new SOS1 inhibitors that are capable of blocking the interaction between SOS1 and Ras-family members, prevent the recycling of KRas in to the active GTP-bound form and, therefore, may provide therapeutic benefit for a wide range of cancers, particularly Ras-associated cancers, SOS1-associated cancers and NF1/NF2-associated cancers.

In one aspect of the invention, compounds are provided represented by Formula (I):

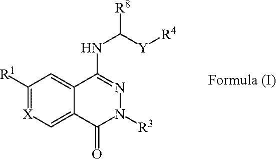

or a pharmaceutically acceptable salt thereof, [0011] wherein:
R¹ is hydrogen, hydroxyl, C1 - C6 alkyl, alkoxy, —N(R⁶)₂, —NR⁶C(O)R⁶, —C(O)N(R⁶)₂, -SO₂alkyl, -SO₂NR⁶alkyl, cycloalkyl, -Q-heterocyclyl, aryl, or heteroaryl, wherein the cycloalkyl, the heterocyclyl, the aryl, and the heteroaryl are each optionally substituted with one or more R²;
each Q is independently a bond, O or ⁶;
X is Nor CR⁷, with the proviso that when X is N, R¹ is not hydroxyl;
each R² is independently hydroxy, halogen, cyano, hydroxyalkyl, haloalkyl, alkoxy, —N(R⁶)₂, -SO₂alkyl, —NR⁶C(O)C1 — C3 alkyl, -C(O)cycloalkyl, -C(O)heretocyclyl or aryl, wherein the cycloalkyl, the heterocyclyl or the aryl are each optionally substituted with one or more R⁹;
R³ is hydrogen, C1 - C3 alkyl, C1 - C3 haloalkyl, or cycloalkyl;
Y is a bond or heteroarylene;
R⁴ is aryl or heteroaryl, each optionally substituted with one or more R⁵;
each R⁵ is independently hydroxy, halogen, cyano, hydroxyalkyl, alkoxy, C1 - C4 alkyl, haloalkyl, —N(R⁶)₂, —L—N(R⁶)₂ or -SO₂alkyl;
L is C1 - C3 alkylene;
each R⁶ is independently hydrogen, C1 - C3 alkyl, haloalkyl or cycloalkyl;
R⁷ is hydrogen, cyano or alkoxy;
R⁸ is C1 - C2 alkyl or haloC1 - C2 alkyl; and
each R⁹ is independently C1 - C3 alkyl or haloalkyl.

In another aspect of the invention, pharmaceutical compositions are provided comprising a therapeutically effective amount of a compound of the present invention or a pharmaceutically acceptable salt thereof and a pharmaceutically acceptable excipient.

In yet another aspect, the invention provides methods for inhibiting the activity of a Ras-family member by inhibiting the associaton between the Ras-family member and SOS1 in a cell, comprising contacting the cell with a compound of Formula (I). In one embodiment, the contacting is in vitro. In one embodiment, the contacting is in vivo.

Also provided herein is a method of inhibiting cell proliferation, in vitro or in vivo, the method comprising contacting a cell with an effective amount of a compound of Formula (I), or a pharmaceutically acceptable salt thereof, or a pharmaceutical composition thereof as defined herein.

Also provided herein are methods for treating cancer in a subject in need thereof, the method comprising (a) determining that cancer is associated with a Ras-family member mutation (e.g., a KRas G12C-associated cancer) (e.g., as determined using a regulatory agency-approved, e.g., FDA-approved, assay or kit); and (b) administering to the patient a therapeutically effective amount of compound of Formula (I), or pharmaceutically acceptable salts or pharmaceutical compositions thereof.

Also provided herein are methods for treating cancer in a subject in need thereof, the method comprising (a) determining that cancer is associated with a SOS1 mutation (e.g., a SOS1-associated cancer) (e.g., as determined using a regulatory agency-approved, e.g., FDA-approved, assay or kit); and (b) administering to the patient a therapeutically effective amount of compound of Formula (I), or pharmaceutically acceptable salts or pharmaceutical compositions thereof.

Also provided herein are methods for treating cancer in a subject in need thereof, the method comprising (a) determining that cancer is associated with a NF-1 or NF-2 mutation (e.g., a NF1/NF2-associated cancer) (e.g., as determined using a regulatory agency-approved, e.g., FDA-approved, assay or kit); and (b) administering to the patient a therapeutically effective amount of compound of Formula (I), or pharmaceutically acceptable salts or pharmaceutical compositions thereof

Also provided herein is a use of a compound of Formula (I), or a pharmaceutically acceptable salt or solvate thereof, as defined herein in the manufacture of a medicament for the inhibition of activity of SOS1.

Also provided herein is the use of a compound of Formula (I), or a pharmaceutically acceptable salt or solvate thereof, as defined herein, in the manufacture of a medicament for the treatment of a SOS1-associated disease or disorder.

### DETAILED DESCRIPTION OF THE INVENTION

The present invention relates to SOS1 inhibitors. In particular, the present invention relates to compounds that inhibit SOS1 activity, pharmaceutical compositions comprising a therapeutically effective amount of the compounds, and methods of use therefor.

### Definitions

Unless defined otherwise, all technical and scientific terms used herein have the same meaning as is commonly understood by one of skill in the art to which this invention belongs. All patents, patent applications, and publications referred to herein are incorporated by reference to the extent they are consistent with the present disclosure. Terms and ranges have their generally defined definition unless expressly defined otherwise.

For simplicity, chemical moieties are defined and referred to throughout primarily as univalent chemical moieties (e.g., alkyl, aryl, etc.). Nevertheless, such terms may also be used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for O, and 2, 4, or 6 for S, depending on the oxidation state of the S).

As used herein, “KRas G12C” refers to a mutant form of a mammalian KRas protein that contains an amino acid substitution of a cysteine for a glycine at amino acid position 12. The assignment of amino acid codon and residue positions for human KRas is based on the amino acid sequence identified by UniProtKB/Swiss-Prot P01116: Variant p.Gly12Cys.

As used herein, “KRas G12D” refers to a mutant form of a mammalian KRas protein that contains an amino acid substitution of an aspartic acid for a glycine at amino acid position 12. The assignment of amino acid codon and residue positions for human KRas is based on the amino acid sequence identified by UniProtKB/Swiss-Prot P01116: Variant p.Gly12Asp.

As used herein, “KRas G12S” refers to a mutant form of a mammalian KRas protein that contains an amino acid substitution of a serine for a glycine at amino acid position 12. The assignment of amino acid codon and residue positions for human KRas is based on the amino acid sequence identified by UniProtKB/Swiss-Prot P01116: Variant p.Gly12Ser.

As used herein, “KRas G12A” refers to a mutant form of a mammalian KRas protein that contains an amino acid substitution of an alanine for a glycine at amino acid position 12. The assignment of amino acid codon and residue positions for human KRas is based on the amino acid sequence identified by UniProtKB/Swiss-Prot P01116: Variant p.Gly12Ala.

As used herein, “KRas G13D” refers to a mutant form of a mammalian KRas protein that contains an amino acid substitution of an aspartic acid for a glycine at amino acid position 13. The assignment of amino acid codon and residue positions for human KRas is based on the amino acid sequence identified by UniProtKB/Swiss-Prot P01116: Variant p.Gly13Asp.

As used herein, “KRas G13C” refers to a mutant form of a mammalian KRas protein that contains an amino acid substitution of a cysteine for a glycine at amino acid position 13. The assignment of amino acid codon and residue positions for human KRas is based on the amino acid sequence identified by UniProtKB/Swiss-Prot P01116: Variant p.Gly13Cys.

As used herein, “KRas Q61L” refers to a mutant form of a mammalian KRas protein that contains an amino acid substitution of a leucine for a glutamine at amino acid position 41. The assignment of amino acid codon and residue positions for human KRas is based on the amino acid sequence identified by UniProtKB/Swiss-Prot P01116: Variant p.Gln61Leu.

As used herein, “KRas A146T” refers to a mutant form of a mammalian KRas protein that contains an amino acid substitution of a threonine for an alanine at amino acid position 146. The assignment of amino acid codon and residue positions for human KRas is based on the amino acid sequence identified by UniProtKB/Swiss-Prot P01116: Variant p.Alal46Thr.

As used herein, “KRas A146V” refers to a mutant form of a mammalian KRas protein that contains an amino acid substitution of a valine for an alanine at amino acid position 146. The assignment of amino acid codon and residue positions for human KRas is based on the amino acid sequence identified by UniProtKB/Swiss-Prot P01116: Variant p.Ala146Val.

As used herein, “KRas A146P” refers to a mutant form of a mammalian KRas protein that contains an amino acid substitution of a proline for an alanine at amino acid position 146. The assignment of amino acid codon and residue positions for human KRas is based on the amino acid sequence identified by UniProtKB/Swiss-Prot P01116: Variant p.Ala146Pro.

As used herein, “HRas G12C” refers to a mutant form of a mammalian HRas protein that contains an amino acid substitution of a cysteine for a glycine at amino acid position 12. The assignment of amino acid codon and residue positions for human HRas is based on the amino acid sequence identified by UniProtKB/Swiss-Prot P01112: Variant p.Gly12Cys.

As used herein, “HRas G12D” refers to a mutant form of a mammalian HRas protein that contains an amino acid substitution of an aspartic acid for a glycine at amino acid position 12. The assignment of amino acid codon and residue positions for human HRas is based on the amino acid sequence identified by UniProtKB/Swiss-Prot P01112: Variant p.Gly12Asp.

As used herein, “HRas G12S” refers to a mutant form of a mammalian HRas protein that contains an amino acid substitution of a serine for a glycine at amino acid position 12. The assignment of amino acid codon and residue positions for human HRas is based on the amino acid sequence identified by UniProtKB/Swiss-Prot P01112: Variant p.Gly12Ser.

As used herein, “HRas G12A” refers to a mutant form of a mammalian HRas protein that contains an amino acid substitution of an alanine for a glycine at amino acid position 12. The assignment of amino acid codon and residue positions for human KRas is based on the amino acid sequence identified by UniProtKB/Swiss-Prot P01112: Variant p.Gly12Ala.

As used herein, “HRas G13D” refers to a mutant form of a mammalian HRas protein that contains an amino acid substitution of an aspartic acid for a glycine at amino acid position 13. The assignment of amino acid codon and residue positions for human HRas is based on the amino acid sequence identified by UniProtKB/Swiss-Prot P01112: Variant p.Gly13Asp.

As used herein, “HRas G13C” refers to a mutant form of a mammalian HRas protein that contains an amino acid substitution of a cysteine for a glycine at amino acid position 13. The assignment of amino acid codon and residue positions for human HRas is based on the amino acid sequence identified by UniProtKB/Swiss-Prot P01112: Variant p.Gly13Cys.

As used herein, “HRas Q61L” refers to a mutant form of a mammalian HRas protein that contains an amino acid substitution of a leucine for a glutamine at amino acid position 41. The assignment of amino acid codon and residue positions for human HRas is based on the amino acid sequence identified by UniProtKB/Swiss-Prot P01112: Variant p.Gln61Leu.

As used herein, “HRas A146T” refers to a mutant form of a mammalian HRas protein that contains an amino acid substitution of a threonine for an alanine at amino acid position 146. The assignment of amino acid codon and residue positions for human NRas is based on the amino acid sequence identified by UniProtKB/Swiss-Prot P01112: Variant p.Ala146Thr.

As used herein, “HRas A146V” refers to a mutant form of a mammalian HRas protein that contains an amino acid substitution of a valine for an alanine at amino acid position 146. The assignment of amino acid codon and residue positions for human NRas is based on the amino acid sequence identified by UniProtKB/Swiss-Prot P01112: Variant p.Ala146Val.

As used herein, “HRas A146P” refers to a mutant form of a mammalian HRas protein that contains an amino acid substitution of a proline for an alanine at amino acid position 146. The assignment of amino acid codon and residue positions for human NRas is based on the amino acid sequence identified by UniProtKB/Swiss-Prot P01112: Variant p.Ala146Pro.

As used herein, “NRas G12C” refers to a mutant form of a mammalian NRas protein that contains an amino acid substitution of a cysteine for a glycine at amino acid position 12. The assignment of amino acid codon and residue positions for human NRas is based on the amino acid sequence identified by UniProtKB/Swiss-Prot P01111: Variant p.Gly12Cys.

As used herein, “NRas G12D” refers to a mutant form of a mammalian NRas protein that contains an amino acid substitution of an aspartic acid for a glycine at amino acid position 12. The assignment of amino acid codon and residue positions for human NRas is based on the amino acid sequence identified by UniProtKB/Swiss-Prot P01111: Variant p.Gly12Asp.

As used herein, “NRas G12S” refers to a mutant form of a mammalian NRas protein that contains an amino acid substitution of a serine for a glycine at amino acid position 12. The assignment of amino acid codon and residue positions for human NRas is based on the amino acid sequence identified by UniProtKB/Swiss-Prot P01111: Variant p.Gly12Ser.

As used herein, “NRas G12A” refers to a mutant form of a mammalian NRas protein that contains an amino acid substitution of an alanine for a glycine at amino acid position 12. The assignment of amino acid codon and residue positions for human KRas is based on the amino acid sequence identified by UniProtKB/Swiss-Prot P01111: Variant p.Gly12Ala.

As used herein, “NRas G13D” refers to a mutant form of a mammalian NRas protein that contains an amino acid substitution of an aspartic acid for a glycine at amino acid position 13. The assignment of amino acid codon and residue positions for human NRas is based on the amino acid sequence identified by UniProtKB/Swiss-Prot P01111: Variant p.Gly13Asp.

As used herein, “HNRas G13C” refers to a mutant form of a mammalian NRas protein that contains an amino acid substitution of a cysteine for a glycine at amino acid position 13. The assignment of amino acid codon and residue positions for human NRas is based on the amino acid sequence identified by UniProtKB/Swiss-Prot P01111: Variant p.Gly13Cys.

As used herein, “HRas Q61L” refers to a mutant form of a mammalian HRas protein that contains an amino acid substitution of a leucine for a glutamine at amino acid position 41. The assignment of amino acid codon and residue positions for human HRas is based on the amino acid sequence identified by UniProtKB/Swiss-Prot P01112: Variant p.Gln61Leu.

As used herein, “NRas A146T” refers to a mutant form of a mammalian NRas protein that contains an amino acid substitution of a threonine for an alanine at amino acid position 146. The assignment of amino acid codon and residue positions for human NRas is based on the amino acid sequence identified by UniProtKB/Swiss-Prot P01111: Variant p.Ala146Thr.

As used herein, “NRas A146V” refers to a mutant form of a mammalian NRas protein that contains an amino acid substitution of a valine for an alanine at amino acid position 146. The assignment of amino acid codon and residue positions for human NRas is based on the amino acid sequence identified by UniProtKB/Swiss-Prot P01111: Variant p.Ala146Val.

As used herein, “NRas A146P” refers to a mutant form of a mammalian NRas protein that contains an amino acid substitution of a proline for an alanine at amino acid position 146. The assignment of amino acid codon and residue positions for human NRas is based on the amino acid sequence identified by UniProtKB/Swiss-Prot P01111: Variant p.Ala146Pro.

As used herein, “a Ras family member” or “Ras family” refers to KRas, HRas, NRas, and activating mutants thereof, including at positions G12, G13, Q61 and A146.

A “Ras family-associated disease or disorder” as used herein refers to diseases or disorders associated with or mediated by or having an activating Ras mutation, such as one at position G12, G13, Q61 or A146. Non-limiting examples of Ras family--associated disease or disorder are a KRas, HRas or NRas G12C-associated cancer, a KRas, HRas or NRas G12D-associated cancer, a KRas, HRas or NRas G12S-associated cancer, a KRas, HRas or NRas G12A-associated cancer, a KRas, HRas or NRas G13D-associated cancer, a KRas, HRas or NRas G13C-associated cancer, a KRas, HRas or NRas Q61 X-associated cancer, a KRas, HRas or NRas A146T-associated cancer, a KRas, HRas or NRas A146V-associated cancer or a KRas, HRas or NRas A146P-associated cancer.

As used herein, “SOS1” refers to a mammalian Son of sevenless homolog 1 (SOS1) enzyme.

A “SOS1-associated disease or disorder” as used herein refers to diseases or disorders associated with or mediated by or having an activating SOS1 mutation. Examples of activating SOS1 mutations include SOS1 N233S and SOS1 N233Y mutations.

As used herein, “SOS1 N233S” refers to a mutant form of a mammalian SOS1 protein that contains an amino acid substitution of a serine for a glutamine at amino acid position 233. The assignment of amino acid codon and residue positions for human SOS1 is based on the amino acid sequence identified by UniProtKB/Swiss-Prot Q07889: Variant p.Gln233Ser.

As used herein, “SOS1 N233Y” refers to a mutant form of a mammalian SOS1 protein that contains an amino acid substitution of a tyrosine for a glutamine at amino acid position 233. The assignment of amino acid codon and residue positions for human SOS1 is based on the amino acid sequence identified by UniProtKB/Swiss-Prot Q07889: Variant p.Gln233Tyr.

As used herein, an “SOS1 inhibitor” refers to compounds of the present invention that are represented by Formula (I) as described herein. These compounds are capable of negatively inhibiting all or a portion of the interaction of SOS1 with Ras family mutant or SOS1 activating mutation thereby reducing and/or modulating the nucleotide exchange activity of Ras family member - SOS1 complex.

As used herein, a “NF-⅟NF-2 -associated disease or disorder” refers to diseases or disorders associated with or mediated by or having a loss-of-function mutation in the neurofibromin (NF-1) gene or neurofibromin 2 (NF-2) gene.

As used herein, a “loss-of-function mutation” refers to any point mutation(s), splice site mutation(s), fusions, nonsense mutations (an amino acid is mutated to a stop codon), in-frame or frame-shifting mutations, including insertions and deletions, and a homozygous deletion of the genes encoding the protein in a target cell or cancer cell that results in a partial or complete loss of the presence, activity and/or function of the encoded protein.

The term “amino” refers to —NH₂.

The term “acetyl” refers to “—C(O)CH₃.

As herein employed, the term “acyl” refers to an alkylcarbonyl or arylcarbonyl substituent wherein the alkyl and aryl portions are as defined herein.

The term “alkyl” as employed herein refers to straight and branched chain aliphatic groups having from 1 to 12 carbon atoms. As such, “alkyl” encompasses C₁, C₂, C₃, C₄, C₅, C₆, C₇, C₈, C₉, C₁₀, C₁₁ and C₁₂ groups. Examples of alkyl groups include, without limitation, methyl, ethyl, propyl, isopropyl, butyl, isobutyl, sec-butyl, tert-butyl, pentyl, and hexyl.

The term “alkenyl” as used herein means an unsaturated straight or branched chain aliphatic group with one or more carbon-carbon double bonds, having from 2 to 12 carbon atoms. As such, “alkenyl” encompasses C₂, C₃, C₄, C₅, C₆, C₇, C₈, C₉, C₁₀, C₁₁ and C₁₂ groups. Examples of alkenyl groups include, without limitation, ethenyl, propenyl, butenyl, pentenyl, and hexenyl.

The term “alkynyl” as used herein means an unsaturated straight or branched chain aliphatic group with one or more carbon-carbon triple bonds, having from 2 to 12 carbon atoms. As such, “alkynyl” encompasses C₂, C₃, C₄, C₅, C₆, C₇, C₈, C₉, C₁₀, C₁₁ and C₁₂ groups. Examples of alkynyl groups include, without limitation, ethynyl, propynyl, butynyl, pentynyl, and hexynyl.

An “alkylene,” “alkenylene,” or “alkynylene” group is an alkyl, alkenyl, or alkynyl group, as defined hereinabove, that is positioned between and serves to connect two other chemical groups. Examples of alkylene groups include, without limitation, methylene, ethylene, propylene, and butylene. Exemplary alkenylene groups include, without limitation, ethenylene, propenylene, and butenylene. Exemplary alkynylene groups include, without limitation, ethynylene, propynylene, and butynylene.

The term “alkoxy” refers to —OC1 — C6 alkyl.

The term “cycloalkyl” as employed herein is a saturated and partially unsaturated cyclic hydrocarbon group having 3 to 12 carbons. As such, “cycloalkyl” includes C₃, C₄, C₅, C₆, C₇, C₈, C₉, C₁₀, C₁₁ and C₁₂ cyclic hydrocarbon groups. Examples of cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” refers to an alkyl group, as defined hereinabove, wherein one or more carbon atoms in the chain are independently replaced O, S, or NR^{x}, wherein R^{x} is hydrogen or C1 - C3 alkyl. Examples of heteroalkyl groups include methoxymethyl, methoxyethyl and methoxypropyl.

An “aryl” group is a C₆-C₁₄ aromatic moiety comprising one to three aromatic rings. As such, “aryl” includes C₆, C₁₀, C₁₃, and C₁₄ cyclic hydrocarbon groups. An exemplary aryl group is a C₆-C₁₀ aryl group. Particular aryl groups include, without limitation, phenyl, naphthyl, anthracenyl, and fluorenyl. An “aryl” group also includes fused multicyclic (e.g., bicyclic) ring systems in which one or more of the fused rings is non-aromatic, provided that at least one ring is aromatic, such as indenyl.

An “aralkyl” or “arylalkyl” group comprises an aryl group covalently linked to an alkyl group wherein the moiety is linked to another group via the alkyl moiety. An exemplary aralkyl group is -(C1 - C6)alkyl(C6 - C10)aryl, including, without limitation, benzyl, phenethyl, and naphthylmethyl.

A “heterocyclyl” or “heterocyclic” group is a mono- or bicyclic (fused or spiro) ring structure having from 3 to 12 atoms, (3, 4, 5, 6, 7, 8, 9, 10, 11 or 12 atoms), for example 4 to 8 atoms, wherein one or more ring atoms are independently —C(O)—, N, NR⁴, O, or S, and the remainder of the ring atoms are quaternary or carbonyl carbons. Examples of heterocyclic groups include, without limitation, epoxy, oxiranyl, oxetanyl, azetidinyl, aziridinyl, tetrahydrofuranyl, tetrahydropyranyl, tetrahydrothiophenyl, pyrrolidinyl, piperidinyl, piperazinyl, imidazolidinyl, thiazolidinyl, thiatanyl, dithianyl, trithianyl, azathianyl, oxathianyl, dioxolanyl, oxazolidinyl, oxazolidinonyl, decahydroquinolinyl, piperidonyl, 4-piperidonyl, thiomorpholinyl, dimethyl-morpholinyl, and morpholinyl. Specifically excluded from the scope of this term are compounds having adjacent ring O and/or S atoms.

As used herein, “heterocyclyl” refers to a heterocyclyl group covalently linked to another group via a bond.

As used herein, the term “heteroaryl” refers to a group having 5 to 14 ring atoms, preferably 5, 6, 10, 13 or 14 ring atoms; having 6, 10, or 14 π electrons shared in a cyclic array; and having, in addition to carbon atoms, from one to three heteroatoms that are each independently N, O, or S. “Heteroaryl” also includes fused multicyclic (e.g., bicyclic) ring systems in which one or more of the fused rings is non-aromatic, provided that at least one ring is aromatic and at least one ring contains an N, O, or S ring atom.

Examples of heteroaryl groups include acridinyl, azocinyl, benzimidazolyl, benzofuranyl, benzo[d]oxazol-2(3H)-one, 2H-benzo[b][1,4]oxazin-3(4H)-one, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

A “heteroaralkyl” or “heteroarylalkyl” group comprises a heteroaryl group covalently linked to another group via a bond. Examples of heteroalkyl groups comprise a C₁- C₆ alkyl group and a heteroaryl group having 5, 6, 9, or 10 ring atoms. Examples of heteroaralkyl groups include pyridylmethyl, pyridylethyl, pyrrolylmethyl, pyrrolylethyl, imidazolylmethyl, imidazolylethyl, thiazolylmethyl, thiazolylethyl, benzimidazolylmethyl, benzimidazolylethyl quinazolinylmethyl, quinolinylmethyl, quinolinylethyl, benzofuranylmethyl, indolinylethyl isoquinolinylmethyl, isoinodylmethyl, cinnolinylmethyl, and benzothiophenylethyl. Specifically excluded from the scope of this term are compounds having adjacent ring O and/or S atoms.

An “arylene,” “heteroarylene,” or “heterocyclylene” group is an bivalent aryl, heteroaryl, or heterocyclyl group, respectively, as defined hereinabove, that is positioned between and serves to connect two other chemical groups.

As employed herein, when a moiety (e.g., cycloalkyl, aryl, heteroaryl, heterocyclyl, urea, etc.) is described as “optionally substituted” without expressly stating the substituents it is meant that the group optionally has from one to four, preferably from one to three, more preferably one or two, non-hydrogen substituents.

The term “halogen” or “halo” as employed herein refers to chlorine, bromine, fluorine, or iodine.

The term “haloalkyl” refers to an alkyl chain in which one or more hydrogens have been replaced by a halogen. Exemplary haloalkyls are trifluoromethyl, difluoromethyl, flurochloromethyl, chloromethyl, and fluoromethyl.

The term “hydroxyalkyl” refers to -alkylene-OH.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a cancer having a KRas G12 or G13 mutation (e.g., as determined using a regulatory agency-approved, e.g., FDA-approved, assay or kit). In some embodiments, the subject has a tumor that is positive for a KRas G12C mutation, a KRas G12D mutation, a KRas G12S mutation, a KRas G12A mutaation, a KRas G13D mutation or a KRas G13C mutation (e.g., as determined using a regulatory agency-approved assay or kit). The subject can be a subject with a tumor(s) that is positive for a a KRas G12C mutation, a KRas G12D mutation, a KRas G12S mutation, a KRas G12A mutaation, a KRas G13D mutation or a KRas G13C mutation (e.g., identified as positive using a regulatory agency-approved, e.g., FDA-approved, assay or kit). The subject can be a subject whose tumors have a KRas G12C mutation, a KRas G12D mutation, a KRas G12S mutation, a KRas G12A mutaation, a KRas G13D mutation or a KRas G13C mutation (e.g., where the tumor is identified as such using a regulatory agency-approved, e.g., FDA-approved, kit or assay). In some embodiments, the subject is suspected of having a KRas G12 or G13 gene-associated cancer. In some embodiments, the subject has a clinical record indicating that the subject has a tumor that has a KRas G12C mutation (and optionally the clinical record indicates that the subject should be treated with any of the compositions provided herein).

The term “pediatric patient” as used herein refers to a patient under the age of 16 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman RE, Kliegman R, Arvin AM, Nelson WE. Nelson Textbook of Pediatrics, 15th Ed. Philadelphia: W.B. Saunders Company, 1996; Rudolph AM, et al. Rudolph’s Pediatrics, 21st Ed. New York: McGraw-Hill, 2002; and Avery MD, First LR. Pediatric Medicine, 2nd Ed. Baltimore: Williams & Wilkins; 1994.

As used herein, “an effective amount” of a compound is an amount that is sufficient to negatively modulate or inhibit the activity of SOS1 enzyme.

As used herein, a “therapeutically effective amount” of a compound is an amount that is sufficient to ameliorate or in some manner reduce a symptom or stop or reverse progression of a condition, or negatively modulate or inhibit the activity of SOS1. Such amount may be administered as a single dosage or may be administered according to a regimen, whereby it is effective.

As used herein, “treatment” means any manner in which the symptoms or pathology of a condition, disorder or disease in a patient are ameliorated or otherwise beneficially altered.

As used herein, “amelioration of the symptoms of a particular disorder by administration of a particular compound or pharmaceutical composition” refers to any lessening, whether permanent or temporary, lasting or transient, that can be attributed to or associated with administration of the composition.

### Compounds

In one aspect of the invention, compounds are provided represented by Formula (I):

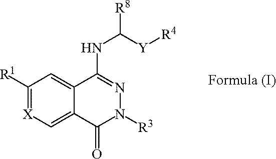

or a pharmaceutically acceptable salt thereof, [0100] wherein:
R¹ is hydrogen, hydroxyl, C1 - C6 alkyl, alkoxy, —N(R⁶)₂, —NR⁶C(O)R⁶, —C(O)N(R⁶)₂, -SO₂alkyl, -SO₂NR⁶alkyl, cycloalkyl, -Q-heterocyclyl, aryl, or heteroaryl, wherein the cycloalkyl, the heterocyclyl, the aryl, and the heteroaryl are each optionally substituted with one or more R²;
each Q is independently a bond, O or NR⁶;
X is N or CR⁷, with the proviso that when X is N, R¹ is not hydroxyl;
each R² is independently hydroxy, halogen, cyano, hydroxyalkyl, haloalkyl, alkoxy, —N(R⁶)₂, -SO₂alkyl, —NR⁶C(O)C1 — C3 alkyl, -C(O)cycloalkyl, -C(O)heretocyclyl or aryl, wherein the cycloalkyl, the heterocyclyl or the aryl are each optionally substituted with one or more R⁹;
R³ is hydrogen, C1 - C3 alkyl, C1 - C3 haloalkyl, or cycloalkyl;
Y is a bond or heteroarylene;
R⁴ is aryl or heteroaryl, each optionally substituted with one or more R⁵
each R⁵ is independently hydroxy, halogen, cyano, hydroxyalkyl, alkoxy, C1 - C4 alkyl, haloalkyl, —N(R⁶)₂, —L—N(R⁶)₂ or -SO₂alkyl;
L is C1 - C3 alkylene;
each R⁶ is independently hydrogen, C1 - C3 alkyl, haloalkyl or cycloalkyl;
R⁷ is hydrogen, cyano or alkoxy;
R⁸ is C1 - C2 alkyl or haloC1 - C2 alkyl; and
each R⁹ is independently C1 - C3 alkyl or haloalkyl.

In one aspect for compounds of Formula (I), X is N. In certain embodiments wherein X is N, R¹ is alkoxy or -Q-heterocyclyl optionally substituted with one or more R². In certain embodiments, wherein X is N, R¹ is -Q-heterocyclyl, and wherein Q is a bond and the heterocyclyl is morpholinyl, piperazinyl, or piperazinone is optionally substituted with one or more R².

In one embodiment for compounds of Formula (I), X is CR⁷. In one embodiment when X is CR⁷, R⁷ is cyano.

In one embodiment for compounds of Formula (I), X is CR⁷. In one embodiment when X is CR⁷, R⁷ is hydrogen.

In one embodiment when X is CR⁷ and R⁷ is hydrogen, R¹ is hydrogen. In another embodiment, R¹ is hydroxyl. In certain embodiments, R¹ is —N(R⁶)₂. In one embodiment, R¹ is —N(R⁶)₂ and each R⁶ is C1 - C3 alkyl. In one embodiment, each C1 - C3 alkyl group is methyl. In other embodiments R¹ is —NR⁶C(O)R⁶. In one embodiment, each C1 - C3 alkyl is methyl. In one embodiment, the R⁶ of the NR⁶ is hydrogen and R⁶ of the C(O)R⁶ is C1 - C3 alkyl.

In another embodiment when X is CR⁷ and R⁷ is hydrogen, R¹ is —C(O)N(R⁶)₂. In one embodiment, each C1 - C3 alkyl is methyl. In one embodiment, each C1 - C3 alkyl is hydrogen. In certain embodiments, R¹ is -SO₂alkyl or -SO₂NR⁶alkyl. In one embodiment, R¹ is -SO₂NR⁶alkyl and R⁶ is hydrogen. In other embodiments, R¹ is cycloalkyl optionally substituted with one or more R². In one embodiment, the cycloalkyl is cyclobutyl, cyclopentyl or cyclohexyl, each optionally substituted with one or more R². In one embodiment, the cyclobutyl, cyclopentyl or the cyclohexyl are substituted with one R², wherein R² is C1 - C3 alkyl, alkoxy, hydroxyl or —N(R⁶)₂. In one embodiment, R² is —N(R⁶)₂ and each R⁶ is C1 - C3 alkyl. In one embodiment, each C1 - C3 alkyl is methyl.

In another embodiment when X is CR⁷ and R⁷ is hydrogen, R¹ is -Q-heterocyclyl optionally substituted with one or more R². In one embodiment, Q is a bond and the heterocyclyl is morpholinyl, piperdinyl, piperazinyl, N-methyl piperazinyl, piperazinone, 1-methyl-piperazin-2-one, or 4-methylthiomorpholine 1,1-dioxide. In another embodiment, Q is a bond and the heterocyclyl is pyrrolidinyl or tetrahydropyranyl, each optionally substituted with one or more R². In one embodiment, the pyrrolidinyl or the tetrahydropyranyl are substituted with one R², wherein R² is C1 - C3 alkyl, alkoxy, hydroxyl or —N(R⁶)₂.

In another embodiment when X is CR⁷ and R⁷ is hydrogen, R¹ is -Q-heterocyclyl, Q is a bond and the heterocyclyl is piperazinyl substituted with one R², wherein R² is -C(O)cycloalkyl or -C(O)heterocyclyl, wherein the cycloalkyl or heterocyclyl portion of the -C(O)cycloalkyl or -C(O)heterocyclyl are each optionally substituted with one or more R⁹. In one embodiment, R² is -C(O)cycloalkyl and the cycloalkyl is cyclopropyl substituted with one R⁹, wherein R⁹ is C1 -C3 alkyl or haloalkyl. In one embodiment, R² is -C(O)heterocyclyl, wherein the heterocyclyl is oxetanyl or tetrahydropyranyl.

In another embodiment when X is CR⁷ and R⁷ is hydrogen, R¹ is -Q-heterocyclyl, Q is a bond and the heterocyclyl is a bicyclic heterocyclyl. In certain embodiments, the bicyclic heterocyclyl is diazabicyclo[3.2.0]heptan-2-yl, (1R,5R)-2,6-diazabicyclo[3.2.0]heptan-2-yl, diazabicyclo[3.2.0]heptan-6-yl, (1R,5R)-2,6-diazabicyclo[3.2.0]heptan-6-yl or (R)-2-methylhexahydropyrrolo[1,2-a]pyrazin-6(2H)-one.

In yet another embodiment, Q is O and the heterocyclyl is azetidinyl, tetrahydrofuranyl, pyrrolidinyl, or piperdinyl.

In yet another embodiment, Q is NR⁶ and the heterocyclyl is tetrahydrofuranyl, pyrrolidinyl, or piperdinyl.

In another embodiment when X is CR⁷ and R⁷ is hydrogen, R¹ is aryl optionally substituted with one or more R². In one embodiment, the aryl is phenyl optionally substituted with one or more R². In certain embodiments, the phenyl is substituted with one R², wherein R² is C1 - C3 alkyl, alkoxy, hydroxyl or —N(R⁶)₂. In one embodiment, R² is —N(R⁶)₂ and each R⁶ is C1 - C3 alkyl. In one embodiment, each C1 - C3 alkyl is methyl. In other embodiments, R¹ is heteroaryl optionally substituted with one or more R². In one embodiment, the heteroaryl is pyrazolyl optionally substituted with one or more R². In one embodiment, the pyrazolyl is substituted with one R², wherein R² is C1 - C3 alkyl, alkoxy, hydroxyl or —N(R⁶)₂. In one embodiment, R² is —N(R⁶)₂ and each R⁶ is C1 - C3 alkyl. In one embodiment, each C1 - C3 alkyl is methyl.

In one embodiment for compounds of Formula (I), X is CR⁷ and R⁷ is alkoxy. In one embodiment, the alkoxy is methoxy. In certain embodiments wherein X is CR⁷ and R⁷ is alkoxy, R¹ is alkoxy. In one embodiment, the alkoxy is methoxy.

In certain embodiments for compounds of Formula (I) wherein X is N or CR⁷, Y is heteroarylene. In one embodiment, the heteroarylene is thiophenylene.

In certain embodiments for compounds of Formula (I) wherein X is N or CR⁷, Y is a bond.

In certain embodiments for compounds of Formula (I), R⁴ is aryl or heteroaryl, each optionally substituted with one or more R⁵. In one embodiment, R⁴ is aryl optionally substituted with one or more R⁵. In one embodiment, the aryl is phenyl optionally substituted with one or more R⁵. In certain embodiments, the phenyl is substituted with one R⁵, wherein R⁵ is C1 - C4 alkyl, haloalkyl, —N(R⁶)₂, —L—N(R⁶)₂ or -SO₂alkyl. In one embodiment, R⁵ is —L—N(R⁶)₂, wherein L is methylene and one R⁶ is hydrogen and the second R⁶ is C1 - C3 alkyl. In one embodiment, the C1 - C3 alkyl is methyl. In another embodiment, R⁵ is —L—N(R⁶)₂, wherein L is methylene and each R⁶ is C1 - C3 alkyl. In one embodiment, each of the C1 - C3 alkyl is methyl.

In certain embodiments wherein R⁴ is aryl, R⁴ is phenyl substituted with two R⁵ wherein one R⁵ is C1 - C4 alkyl and the second R⁵ is haloalkyl. In one embodiment, the C1 -C3 alkyl is methyl and the haloalkyl is trifluoromethyl. In certain embodiments, R⁴ is phenyl substituted with two R⁵, wherein one R⁵ is C1 - C4 alkyl and the second R⁵ is —L—N(R⁶)₂. In one embodiment, L is a methylene and each R⁶ is C1 - C3 alkyl.

In one embodiment for compounds of Formula (I), R³ is hydrogen.

In certain embodiments for compounds of Formula (I), R³ is C1 - C3 alkyl. In one embodiment, the C1 - C3 alkyl is methyl, ethyl or isopropyl.

In certain embodiments for compounds of Formula (I), R³ is cycloalkyl. In one embodiment, the cycloalkyl is cyclopropyl.

In certain embodiments for compounds of Formula (I), R³ is C1 - C3 haloalkyl. In one embodiment, the C1 - C3 haloalkyl is trifluoromethyl, difluoromethyl, fluoromethyl, trifluoroethyl, difluoroethyl, or fluoroethyl.

In certain embodiments for compounds of Formula (I), R⁸ is C1 - C2 alkyl. In one embodiment, the C1 - C2 alkyl is methyl.

In certain embodiments for compounds of Formula (I), R⁸ is haloC1 - C2 alkyl. In one embodiment, the haloC1 - C2 alkyl is fluoromethyl, difluoromethyl or trifluoromethyl.

In one embodiment, the compound of Formula (I) is:

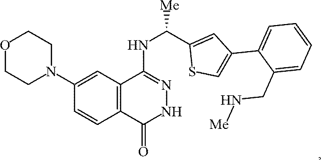

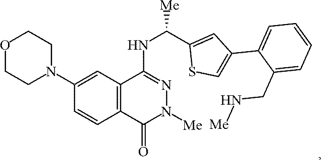

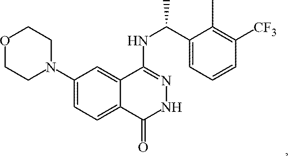

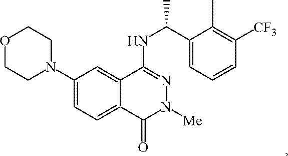

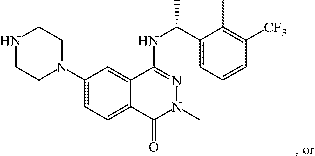

, or

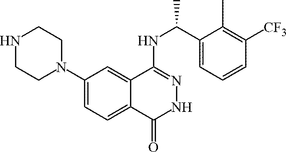 [SMILES: Cc1c([C@@H](C)Nc2n[nH]c(=O)c3ccc(N4CCNCC4)cc23)cccc1C(F)(F)F]

and pharmaceutically acceptable salts of the foregoing compounds.

The compounds of Formula (I) may be formulated into pharmaceutical compositions.

### Pharmaceutical Compositions

In another aspect, the invention provides pharmaceutical compositions comprising a SOS1 inhibitor according to the invention and a pharmaceutically acceptable carrier, excipient, or diluent. Compounds of the invention may be formulated by any method well known in the art and may be prepared for administration by any route, including, without limitation, parenteral, oral, sublingual, transdermal, topical, intranasal, intratracheal, or intrarectal. In certain embodiments, compounds of the invention are administered intravenously in a hospital setting. In certain other embodiments, administration may preferably be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington’s Pharmaceutical Sciences, 18th Edition, ed. A. Gennaro, Mack Publishing Co., Easton, Pa., 1990.

As used herein, the term “pharmaceutically acceptable salts” refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O—alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. A dose of the active compound for all of the above-mentioned conditions is in the range from about 0.01 to 300 mg/kg, preferably 0.1 to 100 mg/kg per day, more generally 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising compounds of the present invention may be used in the methods described herein.

### Methods of Use

In yet another aspect, the invention provides for methods for inhibiting SOS1 activity in a cell, comprising contacting the cell in which inhibition of SOS1 activity is desired in vitro with an effective amount of a compound of Formula (I), pharmaceutically acceptable salts thereof or pharmaceutical compositions containing the compound or pharmaceutically acceptable salt thereof.

The compositions and methods provided herein are particularly deemed useful for inhibiting SOS1 activity in a cell. In one embodiment, a cell in which inhibition of SOS1 activity is desired is contacted in vivo with a therapeutically effective amount of a compound of Formula (I) to negatively modulate the activity of SOS1. In other embodiments, a therapeutically effective amount of pharmaceutically acceptable salt or pharmaceutical compositions containing the compound of Formula (I) may be used. In one embodiment, the cell harbors an activating mutation in a Ras family member, such as KRas, HRas, or NRas. In one embodiment, the cell has aberrant SOS1 activity. In one embodiment, the aberrant SOS1 activity is the result of a SOS1 activating mutation. In one embodiment, the SOS1 activating mutation is a N233S or N233Y mutation. In one embodiment, the cell has aberrant NF-1 or NF-2 activity. In one embodiment, the aberrant NF-1 or NF-2 activity is the result of a NF-1 or NF-2 activating mutation.

By negatively modulating the activity of SOS1, the methods are designed to block the interaction between SOS1 and the Ras family member and increased GTP-loading of RAS proteins thereby decreasing or inhibiting the GTP nucleotide exchange and locking the Ras family member in the GDP-bound, inactive form resulting in the inhibition of downstream Ras-mediated signaling. The cells may be contacted in a single dose or multiple doses in accordance with a particular treatment regimen to affect the desired negative modulation of SOS1.

In another aspect, methods of treating cancer comprising administering to a patient having cancer a therapeutically effective amount of a compound of Formula (I), pharmaceutically acceptable salts thereof or pharmaceutical compositions comprising the compound or pharmaceutically acceptable salts thereof are provided. In one embodiment, the cancer is a Ras family-associated cancer. In one embodiment, the cancer is a SOS 1-associated cancer. In one embodiment, the cancer is a NF-1/NF-2-associated cancer.

The compositions and methods provided herein may be used for the treatment of a wide variety of cancer including tumors such as prostate, breast, brain, skin, cervical carcinomas, testicular carcinomas, etc More particularly, cancers that may be treated by the compositions and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi’s sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm’s tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing’s sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin’s disease, non-Hodgkin’s lymphoma (malignant lymphoma); Skin: malignant melanoma, basal cell carcinoma, squamous cell carcinoma, Kaposi’s sarcoma, moles dysplastic nevi, lipoma, angioma, dermatofibroma, keloids, psoriasis; and Adrenal glands: neuroblastoma. In certain embodiments, the cancer is diffuse large B-cell lymphoma (DLBCL).

In one embodiment, the cancer is a Ras family-associated cancer, such as a KRas, NRas or HRas-associated cancer. In certain embodiments, the Ras family-associated cancer is non-small cell lung cancer or pancreatic cancer. In one embodiment, the cancer is a SOS1-associated cancer. In certain embodiments, the SOS1-associated cancer is lung adenocarcinoma, embryonal rhabdomyosarcoma, Sertoli cell testis tumor and granular cell tumors of the skin. In one embodiment, the cancer is a NF-1/NF-2-associated cancer.

The concentration and route of administration to the patient will vary depending on the cancer to be treated. The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

### GENERAL REACTION SCHEME, INTERMEDIATES AND EXAMPLES

### General Reaction Schemes

The compounds of the present invention may be prepared using commercially available reagents and intermediates in the synthetic methods and reaction schemes described herein, or may be prepared using other reagents and conventional methods well known to those skilled in the art.

The compounds of the present invention may be prepared using commercially available reagents and intermediates in the synthetic methods and reaction schemes described herein, or may be prepared using other reagents and conventional methods well known to those skilled in the art.

For instance, intermediates for preparing compounds and compounds of Formula (I) of the present invention may be prepared according to General Reaction Schemes I - V:

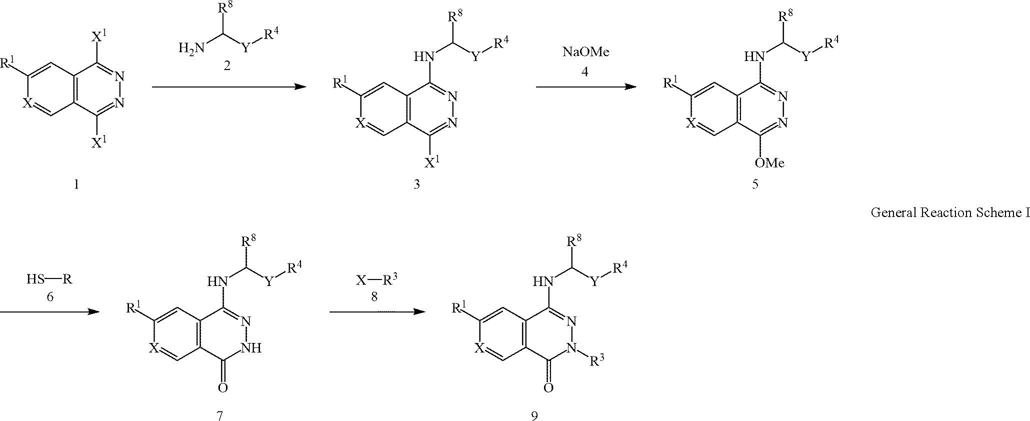

For General Reaction Scheme I, Compounds 7 and 9 are examples of Formula (I). In this General Reaction Scheme I, 1 is reacted with an amine such as intermediate 2, this reaction could for example be a nucleophilic substitution or a metal catalyzed reaction, to yield Compound 3. Compound 3 can then undergo a nucleophilic substitution or metal catalyzed reaction with a coupling partner, such as a sodium methoxide, to form compound 5. Compound 5 can then be treated with a thiol 6 in the presence of a suitable base, e.g., sodium hydride, to form title compound 7. Compound 7 can be further alkylated with an appropriate alkylating agent X-R³ 8 in the presence of a suitable base, such as potassium carbonate, to give the title compound 9.

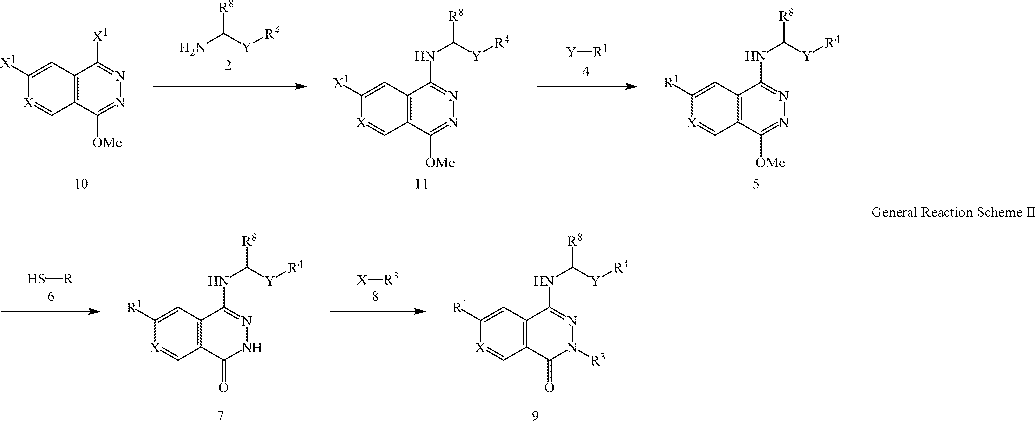

For General Reaction Scheme II, Compounds 7 and 9 are examples of Formula (I). In this General Reaction Scheme II, 10 is reacted with an amine such as intermediate 2, this reaction could for example be a nucleophilic substitution or a metal catalyzed reaction, to yield Compound 11. Compound 11 can then undergo a metal catalyzed reaction with a coupling partner, such as a boronic acid derivative, Y-R¹ 12 in the presence of a suitable base, e.g., sodium carbonate, to form compound 5. Compound 5 can then be treated with a thiol 6 in the presence of a suitable base, e.g., sodium hydride, to form title compound 7. Compound 7 can be further alkylated with an appropriate alkylating agent X-R³ 8 in the presence of a suitable base, such as potassium carbonate, to give the title compound 9.

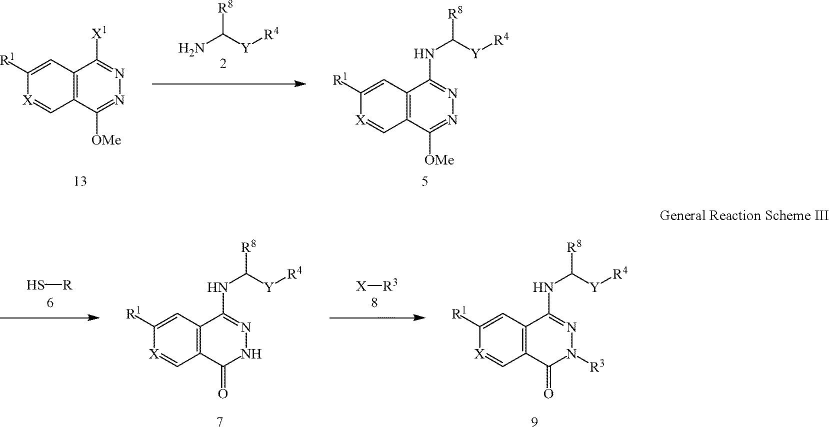

For General Reaction Scheme III, Compounds 7 and 9 are examples of Formula (I). In this General Reaction Scheme III, Compound 13 is reacted with an amine such as intermediate 2, this reaction could for example be a nucleophilic substitution or a metal catalyzed reaction, to form compound 5. Compound 5 can then be treated with a thiol 6 in the presence of a suitable base, e.g., sodium hydride, to form title compound 7. Compound 7 can be further alkylated with an appropriate alkylating agent X-R³ 8 in the presence of a suitable base, such as potassium carbonate, to give the title compound 9.

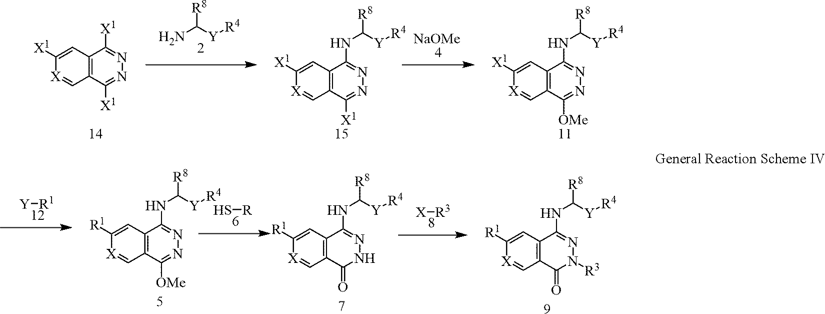

For General Reaction Scheme IV, Compounds 7 and 9 are examples of Formula (I). In this General Reaction Scheme IV, compound 14 is reacted with an amine such as intermediate 2, this reaction could for example be a nucleophilic substitution or a metal catalyzed reaction, to yield compound 15. Compound 15 can then undergo a nucleophilic substitution or metal catalyzed reaction with a coupling partner, such as a sodium methoxide, to form compound 5. Compound 5 can then be treated with a thiol 6 in the presence of a suitable base, e.g., sodium hydride, to form title compound 7. Compound 7 can be further alkylated with an appropriate alkylating agent X-R³ 8 in the presence of a suitable base, such as potassium carbonate, to give the title compound 9.

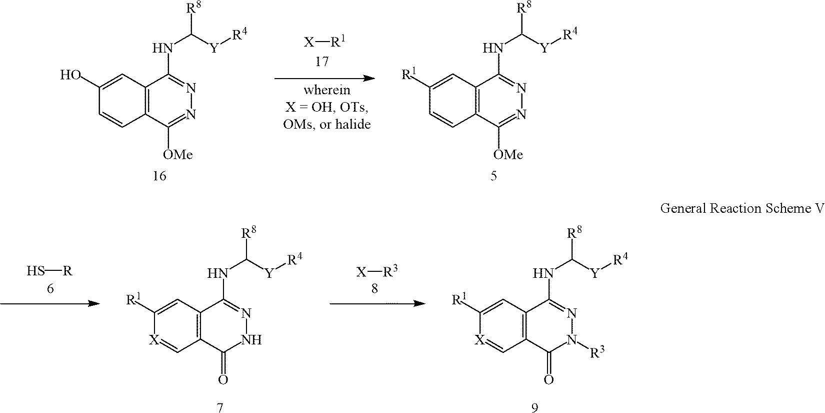

For General Reaction Scheme V, Compounds 7 and 9 are examples of Formula (I). In this General Reaction Scheme V, Compound 16 can participate in a substitution reaction with a coupling partner, such as an alcohol, halide, tosylate, or mesylate X-R¹ 17 in the presence of a suitable base or coupling partner, e.g., cesium carbonate or diethyl azodicarboxylate, to form compound 5. Compound 5 can then be treated with a thiol 6 in the presence of a suitable base, e.g., sodium hydride, to form title compound 7. Compound 7 can be further alkylated with an appropriate alkylating agent X-R³ 8 in the presence of a suitable base, such as potassium carbonate, to give the title compound 9.

The following intermediates may be used to prepare compounds of the present invention.

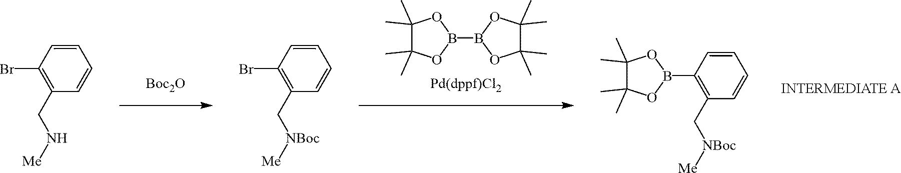

Step A: To a mixture of 1-(2-bromophenyl)-N-methylmethanamine (6.50 g, 32.5 mmol, 1 eq.) in THF (70.0 mL) was added Boc₂O (7.80 g, 35.7 mmol, 8.21 mL, 1.10 eq.) dropwise at 25° C., and the mixture was stirred at 25° C. for 1 hour. The reaction mixture was directly concentrated in vacuo to give a residue. The residue was purified by column chromatography (SiO₂, petroleum ether/ethyl acetate = 20/1 to 10/1) to give tert-butyl (2-bromobenzyl)(methyl)carbamate (7.50 g, 25.0 mmol, 76.9% yield) as a colorless oil.

¹H NMR (400 MHz, CDCl₃) δ 7.55 (br d, J = 8.0 Hz, 1H), 7.34 - 7.28 (m, 1H), 7.22 -7.08 (m, 2H), 4.61 - 4.42 (m, 2H), 2.94 - 2.78 (m, 3H), 1.60 - 1.33 (m, 9H).

Step B: A mixture of tert-butyl (2-bromobenzyl)(methyl)carbamate (7.00 g, 23.3 mmol, 1.00 eq.), bis(pinacolato)diboron (8.88 g, 35.0 mmol, 1.50 eq.), Pd(dppf)Cl₂ (1.71 g, 2.33 mmol, 0.10 eq.) and potassium acetate (5.72 g, 58.3 mmol, 2.50 eq.) in dioxane (80.0 mL) was degassed and purged with nitrogen for 3 times, and then the mixture was stirred at 110° C. for 12 hours under a nitrogen atmosphere. The reaction mixture was concentrated under reduced pressure to give a residue, and the residue was purified by column chromatography (SiO₂, petroleum ether/ethyl acetate = 1/0 to 10/1) to give tert-butyl methyl(2-(4,4,5,5-tetramethyl-1,3,2-dioxaborolan-2-yl)benzyl)carbamate (8.00 g, 23.0 mmol, 98.8% yield) as a colorless oil.

¹H NMR (400 MHz, CDCl₃) δ 7.82 (br d, J = 7.2 Hz, 1H), 7.48 - 7.37 (m, 1H), 7.27 -7.21 (m, 2H), 4.85 - 4.63 (m, 2H), 2.92 - 2.73 (m 3H), 1.54 - 1.41 (m, 9H), 1.35 (s, 12H).

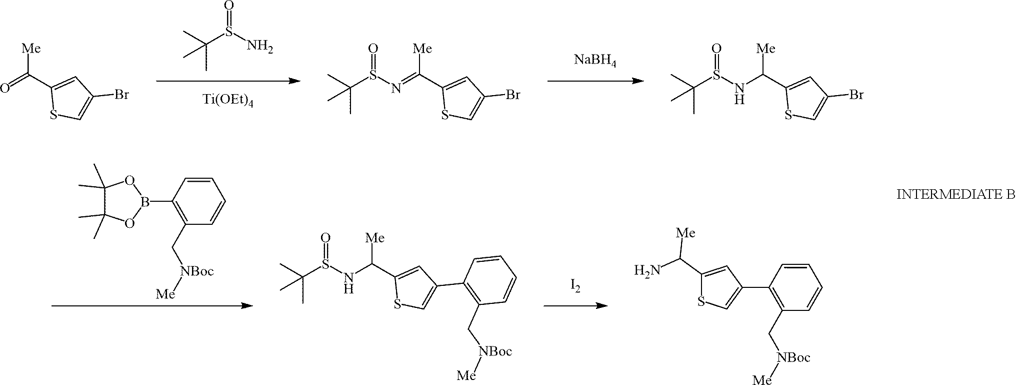

Step A: To a solution of 1-(4-bromothiophen-2-yl)ethan-1-one (4.00 g, 19.5 mmol, 1.10 eq.) and 2-methylpropane-2-sulfinamide (2.15 g, 17.7 mmol, 1.00 eq.) in THF (56.0 mL) was added Ti(OEt)₄ (8.09 g, 35.5 mmol, 7.35 mL, 2.00 eq.). The mixture was stirred at 70° C. for 2 hours. The mixture was poured into water (15.0 mL) and stirred for 5 minutes. The suspension was filtered, and filtrate was concentrated in vacuo to give a residue. The residue was washed with petroleum ether/ethyl acetate= 5/1 (10 mL), filtered, and filter cake was collected and dried in vacuo to give N-(1-(4-bromothiophen-2-yl)ethylidene)-2-methylpropane-2-sulfinamide (3.00 g, 9.73 mmol, 54.9% yield) as a yellow solid.

¹HNMR (400 MHz, CDCl₃) δ 7.43 (d, J = 1.2 Hz, 1H), 7.41 (d, J = 1.2 Hz, 1H), 2.72 (s, 3H), 1.30 (s, 9H).

Step B: To a solution of N-(1-(4-bromothiophen-2-yl)ethylidene)-2-methylpropane-2-sulfinamide (3.70 g, 12.0 mmol, 1.00 eq.) in THF (40.0 mL) was added sodium borohydride (1.36 g, 36.0 mmol, 3.00 eq.) at 0° C. The reaction mixture was warmed slowly to 25° C. and stirred for 2 hours. The mixture was poured into ice-water (15.0 mL) and stirred for 5 minutes at 0° C. The aqueous phase was extracted with ethyl acetate (30.0 mL × 3). The combined organic phases were washed with brine (30.0 mL × 3), dried over anhydrous sodium sulfate, filtered, and concentrated in vacuo to give N-(1-(4-bromothiophen-2-yl)ethyl)-2-methylpropane-2-sulfinamide (3.60 g, 9.51 mmol, 79.3% yield, 82.0% purity) as yellow oil.

¹HNMR (400 MHz, CDCl₃) δ 7.15 (s, 1H), 6.98 - 6.96 (s, 1H), 4.81 - 4.75 (m, 1H), 3.55 (br d, J = 3.6 Hz, 1H), 1.59 (d, J = 6.4 Hz, 3H), 1.24 (s, 9H).

Step C: To a solution of N-(1-(4-bromothiophen-2-yl)ethyl)-2-methylpropane-2-sulfinamide (3.00 g, 9.67 mmol, 1.00 eq.) and tert-butyl methyl(2-(4,4,5,5-tetramethyl-1,3,2-dioxaborolan-2-yl)benzyl)carbamate (5.04 g, 14.5 mmol, 1.50 eq.) in dioxane (35.0 mL) and water (8.00 mL) was added Pd(PPh₃)₄ (1.12 g, 967 µmol, 0.10 eq.) and cesium carbonate (9.45 g, 29.01 mmol, 3.00 eq.) under a nitrogen atmosphere. The mixture was stirred at 110° C. for 2 hours under a nitrogen atmosphere. The mixture was filtered, and the filtrate was concentrated in vacuo to give a residue. The residue was purified by column chromatography (SiO₂, petroleum ether/ethyl acetate = 10/1 to 1/1) to give tert-butyl (2-(5-(1-((tert-butylsulfinyl)amino)ethyl)thiophen-3-yl)benzyl)(methyl)carbamate (1.40 g, 3.11 mmol, 32.1% yield) as yellow oil. LCMS [M+1]: 451.2.

Step D: To a solution of tert-butyl (2-(5-(1-((tert-butylsulfinyl)amino)ethyl)thiophen-3-yl)benzyl)(methyl)carbamate (1.40 g, 4.88 mmol, 1.00 eq.) in THF (15.0 mL) and water (5.00 mL) was added iodine (232 mg, 1.46 mmol, 295 µL, 0.30 eq.). The mixture was stirred at 50° C. for 30 minutes. The residue was poured into saturated sodium sulfite aqueous solution (30.0 mL) and stirred for 5 minutes. The aqueous phase was extracted with ethyl acetate (15.0 mL × 2). The combined organic phases were washed with brine (30.0 mL × 2), dried over anhydrous sodium sulfate, filtered and concentrated in vacuo to give tert-butyl (2-(5-(1-aminoethyl)thiophen-3-yl)benzyl)(methyl)carbamate (1.20 g, crude) as yellow oil.

¹H NMR (400 MHz, CDC1₃) δ 7.36 - 7.28 (m, 3H), 7.26 - 7.22 (m, 1H), 7.01 (s, 1H), 6.91 (br s, 1H), 4.49 (br d, J = 19.2 Hz, 2H), 4.40 (q, J = 6.4 Hz, 1H), 2.72 (br d, J = 19.2 Hz, 3H), 1.53 (d, J = 6.4 Hz, 3H), 1.51 - 1.40 (m, 9H).

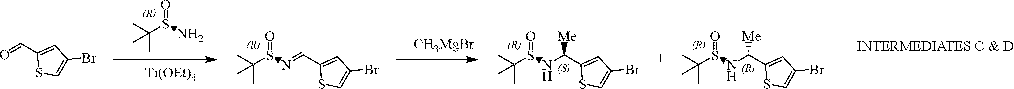

Step A: To a solution of 4-bromothiophene-2-carbaldehyde (20.0 g, 104 mmol, 1.00 eq.) and (R)-2-methylpropane-2-sulfinamide (12.1 g, 99.5 mmol, 0.95 eq.) in THF (200 mL) was added titanium (IV) ethoxide (47.8 g, 209 mmol, 43.4 mL, 2.00 eq.). The reaction mixture was stirred at 25° C. for 1 hour. The mixture was then poured into water (20.0 mL) and stirred for 5 minutes to give a suspension. The suspension was filtered and the filtered liquor was concentrated in vacuo to give (R,E)-N-((4-bromothiophen-2-yl)methylene)-2-methylpropane-2-sulfinamide (20.0 g, crude) as yellow oil. LCMS [M+1]: 295.8.

Step B: To a solution of (R,E)-N-((4-bromothiophen-2-yl)methylene)-2-methylpropane-2-sulfinamide (600 mg, 2.04 mmol, 1.00 eq.) in THF (200 mL) was added methyl magnesium bromide (3.00 M, 2.04 mL, 3.00 eq.) dropwise at 0° C. Then the reaction mixture was stirred at 25° C. for 1 hour. Saturated ammonium chloride aqueous solution (3.00 mL) was added to the reaction mixture and stirred for 5 minutes. The aqueous phase was extracted with ethyl acetate (3.00 mL × 2), and the combined organic phases were washed with brine (3.00 mL × 2), dried over anhydrous sodium sulfate, filtered and concentrated in vacuo to give a residue. The residue was purified by prep-TLC (SiO₂, petroleum ether/ethyl acetate = 1/1) to give (R)-N-((S)-1-(4-bromothiophen-2-yl)ethyl)-2-methylpropane-2-sulfinamide (first eluting, Intermediate C) (120 mg, 19.0% yield) as yellow oil and (R)-N-((R)-1-(4-bromothiophen-2-yl)ethyl)-2-methylpropane-2-sulfinamide (2^{nd} eluting, Intermediate D) (150 mg, 483 µmol, 23.7% yield) as yellow oil.

Intermediate C: ¹H NMR (400 MHz, CDCl₃) δ 7.15 (d, J = 1.6 Hz, 1H), 6.97 (s, 1H), 4.81 - 4.75 (m, 1H), 3.51 (br d, J = 3.2 Hz, 1H), 1.59 (d, J = 6.8 Hz, 3H), 1.24 (s, 9H).

Intermediate D: ¹H NMR (400 MHz, CDC1₃) δ 7.14 (d, J = 1.6 Hz, 1H), 6.89 (s, 1H), 4.81 - 4.74 (m, 1H), 3.39 (br d, J = 5.6 Hz, 1H), 1.65 (d, J = 6.8 Hz, 3H), 1.25 (s, 9H).

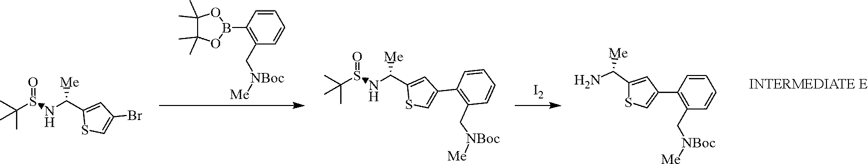 [SMILES: C[C@@H](N[S@](=O)C(C)(C)C)c1cc(Br)cs1 ; CN(Cc1ccccc1B1OC(C)(C)C(C)(C)O1)C(=O)OC(C)(C)C ; C[C@@H](N[S@](=O)C(C)(C)C)c1cc(-c2ccccc2CN(C)C(=O)OC(C)(C)C)cs1 ; "I2" ; C[C@@H](N)c1cc(-c2ccccc2CN(C)C(=O)OC(C)(C)C)cs1]

Step A: To a solution of (R)-N-((R)-1-(4-bromothiophen-2-yl)ethyl)-2-methylpropane-2-sulfinamide (150 mg, 483 µmol, 1.00 eq.) and tert-butyl methyl(2-(4,4,5,5-tetramethyl-1,3,2-dioxaborolan-2-yl)benzyl)carbamate (168 mg, 483 µmol, 1.00 eq.) in dioxane (1.00 mL) and water (0.20 mL) was added Pd(PPh₃)₄ (55.9 mg, 48.3 µmol, 0.10 eq) and cesium carbonate (473 mg, 1.45 mmol, 3.00 eq.) under a nitrogen atmosphere. The reaction mixture was stirred at 110° C. for 2 hours under a nitrogen atmosphere, then to 25° C. and concentrated in vacuo to give a residue. The residue was purified by prep-TLC (SiO₂, petroleum ether/ethyl acetate = 1/1) to give tert-butyl (2-(5-((R)-1-(((R)-tert-butylsulfinyl)amino)ethyl)thiophen-3-yl)benzyl)(methyl)carbamate (120 mg, 266 µmol, 55.1% yield) as a white solid. LCMS [M+1] = 451.1.

¹H NMR (400 MHz, CDCl₃) δ 7.37 - 7.29 (m, 3H), 7.25 (s, 1H), 7.06 (s, 1H), 6.95 (br s, 1H), 4.88 - 4.81 (m, 1H), 4.48 (br d, J = 16.0 Hz, 2H), 3.44 (br d, J = 6.0 Hz, 1H), 2.73 (br d, J= 12.8 Hz, 3H), 1.71 (d, J = 6.4 Hz, 3H), 1.27 (s, 9H), 1.25 (s, 9H).

Step B: To a solution of tert-butyl (2-(5-((R)-1-(((R)-tert-butylsulfinyl)amino)ethyl)thiophen-3-yl)benzyl)(methyl)carbamate (120 mg, 266 µmol, 1.00 eq.) in THF (1.00 mL) and water (0.20 mL) was added iodine (20.3 mg, 79.9 µmol, 16.1 µL, 0.30 eq.), and the reaction mixture was stirred at 50° C. for 1 hour. The reaction mixture was then cooled to 25° C., poured into saturated sodium sulfite aqueous solution (2.00 mL) and stirred for 5 minutes. The aqueous phase was extracted with ethyl acetate (3.00 mL × 3), and the combined organic phases were washed with brine (3.00 mL × 3), dried over anhydrous sodium sulfate, filtered, and concentrated in vacuo to give a residue. The residue was purified by prep-HPLC (column: Phenomenex Gemini-NX C18 75 × 30 mm × 3 um; mobile phase: [water(0.1%TFA)-ACN]; B%: 28% - 38%) to give tert-butyl (R)-(2-(5-(1-aminoethyl)thiophen-3-yl)benzyl)(methyl)carbamate (40.0 mg, 113 µmol, 42.3% yield, 97.5% purity) as white oil.

¹HNMR (400 MHz, CD₃OD) δ 7.41 - 7.23 (m, 6H), 4.84 - 4.79 (m, 1H), 4.48 (s, 2H), 2.73 (s, 3H), 1.76 (d, J = 6.8 Hz, 3H), 1.51 - 1.36 (m, 9H).

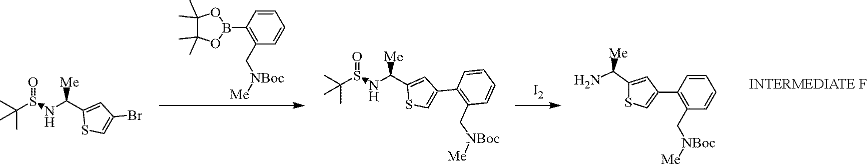 [SMILES: C[C@H](N[S@](=O)C(C)(C)C)c1cc(Br)cs1 ; CN(Cc1ccccc1B1OC(C)(C)C(C)(C)O1)C(=O)OC(C)(C)C ; C[C@H](N[S@](=O)C(C)(C)C)c1cc(-c2ccccc2CN(C)C(=O)OC(C)(C)C)cs1 ; "I2" ; C[C@H](N)c1cc(-c2ccccc2CN(C)C(=O)OC(C)(C)C)cs1]

Step A: To a solution of (R)-N-((S)-1-(4-bromothiophen-2-yl)ethyl)-2-methylpropane-2-sulfinamide (100 mg, 322 µmol, 1.00 eq.) and tert-butyl methyl(2-(4,4,5,5-tetramethyl-1,3,2-dioxaborolan-2-yl)benzyl)carbamate (112 mg, 322 µmol, 1.00 eq.) in dioxane (1.00 mL) and water (0.20 mL) was added Pd(PPh₃)₄ (37.2 mg, 32.2 µmol, 0.10 eq.) and cesium carbonate (315 mg, 967 ummol, 3.00 eq.) under a nitrogen atmosphere. The reaction mixture was stirred at 110° C. for 2 hours, then cooled to 25° C. and concentrated in vacuo to give a residue. The residue was purified by prep-TLC (SiO₂, petroleum ether/ethyl acetate = 1/1) to give tert-butyl (2-(5-((S)-1-(((R)-tert-butylsulfinyl)amino)ethyl)thiophen-3-yl)benzyl)(methyl)carbamate (100 mg, 266 µmol, 68.9% yield) as yellow oil. LCMS [M+1] = 451.1.

¹HNMR (400 MHz, CDCl₃) δ 7.37 - 7.28 (m, 3H), 7.26 - 7.22 (m, 1H), 7.07 (d, J = 1.2 Hz, 1H), 7.03 (br s, 1H), 4.90 - 4.83 (m, 1H), 4.55 - 4.41 (m, 2H), 3.71 - 3.55 (m, 1H), 2.80 - 2.65 (m, 3H), 1.64 (d, J = 6.8 Hz, 3H), 1.52 - 1.41 (m, 9H), 1.26 (s, 9H).

Step B: To a solution of tert-butyl (2-(5-((S)-1-(((R)-tert-butylsulfinyl)amino)ethyl)thiophen-3-yl)benzyl)(methyl)carbamate (100 mg, 266 µmol, 1.00 eq.) in THF (1.00 mL) and water (0.20 mL) was added iodine (16.9 mg, 66.6 µmol, 13.4 µL, 0.30 eq.). The reaction mixture was stirred at 50° C. for 1 hour, thens cooled to 25° C. and poured into saturated aqueous sodium sulfite (2.00 mL) solution and stirred for 5 minutes. The aqueous phase was extracted with ethyl acetate (3.00 mL × 3), and the combined organic phases were washed with brine (3.00 mL × 3), dried over anhydrous sodium sulfate, filtered, and concentrated in vacuo to give a residue. The residue was purified by prep-HPLC (column: Phenomenex Luna C18 150 × 25 mm × 10 um; mobile phase: [water(0.1%TFA)-ACN]; B%: 24%-54%) to give tert-butyl (S)-(2-(5-(1-aminoethyl)thiophen-3-yl)benzyl)(methyl)carbamate (45.0 mg, 97.7 µmol, 44.0% yield, TFA salt) as white oil. LCMS [M+1] = 347.2.

¹HNMR (400 MHz, CD₃OD) δ 7.40 (d, J = 1.2 Hz, 1H), 7.38 - 7.22 (m, 5H), 4.82 -4.80 (br s, 1H), 4.48 (s, 2H), 2.73 (s, 3H), 1.75 (d, J = 6.8 Hz, 3H), 1.50 - 1.35 (m, 9H).

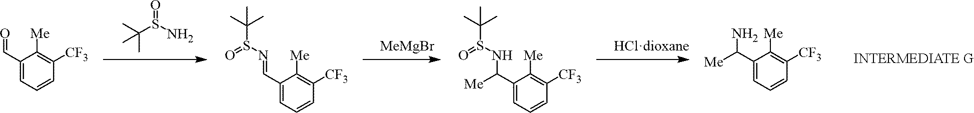

Step A: To a solution of 2-methyl-3-(trifluoromethyl)benzaldehyde (300 mg, 1.59 mmol, 1.00 eq.) and 2-methylpropane-2-sulfinamide (213 mg, 1.75 mmol, 1.10 eq.) in THF (5.00 mL) was added titanium (IV) ethoxide (727 mg, 3.19 mmol, 661 µL,2.00 eq). The reaction mixture was stirred at 25° C. for 12 hours. The reaction mixture was poured into water (2.00 mL) and stirred for 5 minutes to give a suspension. The suspension was filtered and concentrated in vacuo to give 2-methyl-N-(2-methyl-3-(trifluoromethyl)benzylidene)propane-2-sulfinamide (360 mg, 1.24 mmol, 77.5% yield) as a white solid.

¹H NMR (400 MHz, CDCl₃) δ = 8.98 (s, 1H), 8.13 (d, J = 7.6 Hz, 1H), 7.78 (d, J = 7.6 Hz, 1H), 7.40 (t, J= 7.6 Hz, 1H), 2.70 (d, J= 0.8 Hz, 3H), 1.29 (s, 9H).

Step B: To a solution of 2-methyl-N-(2-methyl-3-(trifluoromethyl)benzylidene)propane-2-sulfinamide (185 mg, 635 µmol, 1.00 eq.) in THF (5.00 mL) was added dropwise methyl magnesium bromide (227 mg, 3.00 M, 635 µL, 3.00 eq.) at 0° C. under a nitrogen atmosphere. The reaction mixture was stirred at 25° C. for 3 hours then treated with saturated ammonium chloride solution (10.0 mL) slowly. The organic layer and aqueous phase were separated, and the aqueous phase was extracted with ethyl acetate (5.00 mL × 3). The combined organic layers were washed with brine (10.0 mL), dried over anhydrous sodium sulfate, filtered, and concentrated to give a residue. The residue was purified by column chromatography (SiO₂, petroleum ether/ethyl acetate= 10/1 to 1/1) to give 2-methyl-N-(1-(2-methyl-3-(trifluoromethyl)phenyl)ethyl)propane-2-sulfinamide (150 mg, 488.0 µmol, 76.8% yield) as a yellow solid.

¹H NMR (400 MHz, CDCl₃) δ = 7.65 - 7.54 (m, 4H), 7.35 - 7.28 (m, 2H), 5.00 - 4.87 (m, 2H), 2.49 (s, 6H), 1.54 - 1.50 (m, 6H), 1.26 - 1.24 (m, 9H), 1.22 (s, 9H).

Step C: To a solution of 2-methyl-N-(1-(2-methyl-3-(trifluoromethyl)phenyl)ethyl)propane-2-sulfinamide (150 mg, 488.0 µmol, 1.00 eq.) in HC1 (4.0 M in dioxane, 1.00 mL) was stirred at 25° C. for 1 hour. The reaction mixture was filtered and filter cake was concentrated in vacuo to give 1-(2-methyl-3-(trifluoromethyl)phenyl)ethan-1-amine (45.0 mg, 38.5% yield) as a red solid. LCMS [M+1] = 204.3.

¹H NMR (400 MHz, CD₃OD) δ = 7.78 - 7.65 (m, 2H), 7.56 - 7.48 (m, 1H), 4.93 - 4.89 (m, 1H), 2.52 (d, J = 0.8 Hz, 3H), 1.63 (d, J = 6.8 Hz, 3H).

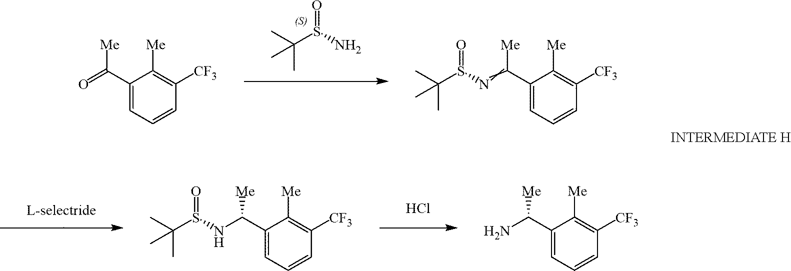

Step A: To a solution of 1-(2-methyl-3-(trifluoromethyl)phenyl)ethan-1-one (8.00 g, 39.6 mmol, 1.00 eq.) and (S)-2-methylpropane-2-sulfinamide (5.28 g, 43.5 mmol, 1.10 eq.) in THF (80.0 mL) was added titanium (IV) ethoxide (18.1 g, 79.1 mmol, 16.4 mL, 2.00 eq.). The reaction mixture was stirred at 70° C. for 2 hours. The reaction mixture was cooled at 25° C. and poured into ice-water (w/w = 1/1) (80.0 mL) and stirred for 15 minutes to give a suspension. The suspension was filtered, the filtrate was extracted with ethyl acetate (50.0 mL × 3). The combined organic phases were washed with brine (30.0 mL × 3), dried over anhydrous sodium sulfate, filtered, and concentrated in vacuo to give a residue. The residue was purified by column chromatography (SiO₂, petroleum ether/ethyl acetate= 20/1 to 3/1) to give (S)-2-methyl-N-(1-(2-methyl-3-(trifluoromethyl)phenyl)ethylidene)propane-2-sulfinamide (8.00 g, 26.2 mmol, 66.2% yield) as yellow oil. LCMS [M+1]: 306.2.

¹H NMR (400 MHz, CD₃OD) δ 7.74 (br t, J = 7.2 Hz, 2H), 7.57 - 7.51 (m, 1H), 7.46 (br t, J= 7.6 Hz, 2H), 7.43 - 7.30 (m, 1H), 2.72 (s, 3H), 2.54 (J = 6.8 Hz, 3H), 2.48 (s, 3H), 2.40 (br d, J= 16.0 Hz, 3H), 1.31 (s, 9H), 1.24 (br d, J = 12.4 Hz, 9H).

Step B: To a solution of S)-2-methyl-N-(1-(2-methyl-3-(trifluoromethyl)phenyl)ethylidene)propane-2-sulfinamide (8.00 g, 26.2 mmol, 1.00 eq.) in THF (80.0 mL) was added L-selectride (7.47 g, 39.3 mmol, 8.59 mL, 1.50 eq.) dropwise at -78° C. The reaction mixture was stirred at -78° C. for 2 hours. Water was added dropwise to the reaction mixture (10.0 mL) at 0° C. and the resulting mixture was stirred for 5 minutes. The aqueous phase was extracted with ethyl acetate (30.0 mL × 3). The combined organic phases were washed with brine (30.0 mL × 2), dried over anhydrous sodium sulfate, filtered, and concentrated in vacuo to give a residue. The residue was purified by column chromatography (SiO₂, petroleum ether/ethyl acetate = 20/1 to 3/1) to give (5)-2-methyl-N-((R)-1-(2-methyl-3-(trifluoromethyl)phenyl)ethyl)propane-2-sulfinamide (3.50 g, 11.4 mmol, 43.5% yield) as yellow oil. LCMS [M+1]: 308.0.

¹H NMR (400 MHz, CD₃OD) δ = 7.70 (d, J= 8.0 Hz, 1H), 7.57 (d, J=7.6 Hz, 1H), 7.39 - 7.33 (m, 1H), 4.94 - 4.88 (m, 1H), 2.48 (d, J = 1.2 Hz, 3H), 1.54 (d, J = 6.4 Hz, 3H), 1.20 (s, 9H).

Step C: A solution of ((R)-1-(2-methyl-3-(trifluoromethyl)phenyl)ethyl)propane-2-sulfinamide (1.30 g, 4.23 mmol, 1.00 eq.) in HC1 (4 M in dioxane, 15.0 mL) was stirred at 25° C. for 30 minutes. The reaction mixture was filtered and filter cake dried in vacuo to give (R)-1-(2-methyl-3-(trifluoromethyl)phenyl)ethan-1-amine (700 mg, 2.89 mmol, 68.4% yield, 99.1 % purity, hydrochloride) as a white solid. LCMS [M+H]: 204.0.

¹H NMR (400 MHz, CD₃OD) δ = 7.73 (t, J= 7.6 Hz, 2H), 7.54 - 7.49 (m, 1H), 4.92 -4.88 (m, 1H), 2.52 (d, J = 0.8 Hz, 3H), 1.62 (d, J = 6.8 Hz, 3H).

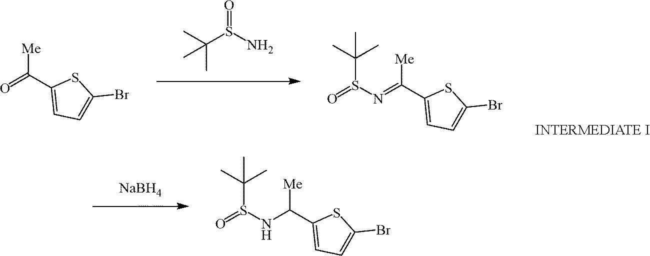

Step A: To a solution of 1-(5-bromothiophen-2-yl)ethan-1-one (11.0 g, 53.6 mmol, 1.00 eq.) in THF (120 mL) was added 2-methylpropane-2-sulfinamide (8.45 g, 69.7 mmol, 1.30 eq.) and titanium (IV) ethoxide (24.5 g, 107 mmol, 22.3 mL, 2.00 eq.), the reaction mixture was stirred at 75° C. for 12 hours under a nitrogen atmosphere. The reaction mixture was cooled to 25° C. and concentrated in vacuo to give a residue, the residue was diluted with water (200 mL) and ethyl acetate (200 mL), filtered, and the filtrate was extracted with ethyl acetate (100 mL × 3). The combined organic layers were washed with brine (300 mL), dried over anhydrous sodium sulfate, filtered, and concentrated under reduced pressued to give N-(1-(5-bromothiophen-2-yl)ethylidene)-2-methylpropane-2-sulfinamide (16.0 g, crude) as a yellow solid. LCMS [M+1]: 308.0.

Step B: To a solution of N-(1-(5-bromothiophen-2-yl)ethylidene)-2-methylpropane-2-sulfinamide (16.0 g, 51.9 mmol, 1.00 eq.) in THF (150 mL) was added sodium borohydride (3.93 g, 104 mmol, 2.00 eq.) at 0° C., the reaction mixture was stirred at 20° C. for 1 hour. Saturated sodium bicarbonate aqueous solution (20.0 mL) was added to the reaction mixture dropwise, then the mixture was diluted with water (200 mL) and extracted with ethyl acetate (100 mL × 3). The combined organic layers were dried over anhydrous sodium sulfate, filtered, and concentrated in vacuo to give a residue. The residue was purified by column chromatography (SiO₂, petroleum ether/ethyl acetate = 30/1 to 2/1) to give N-(1-(5-bromothiophen-2-yl)ethyl)-2-methylpropane-2-sulfinamide (12.0 g, 38.7 mmol, 74.5% yield) as a yellow oil. LCMS [M+1]: 309.9.

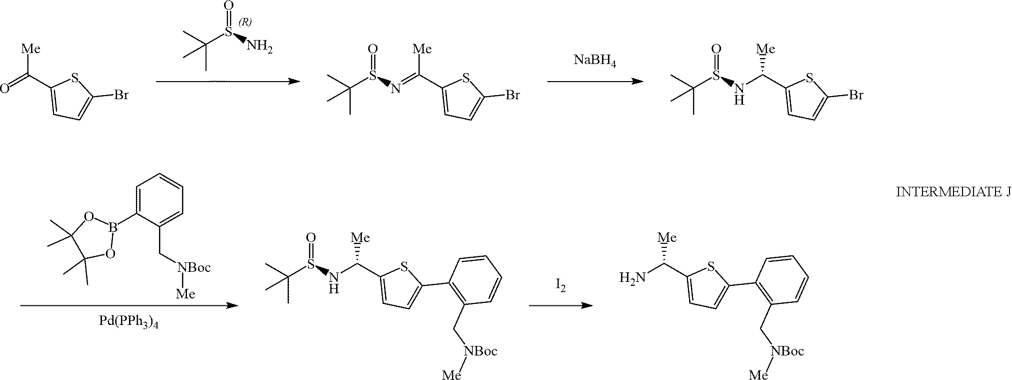

Step A: To a solution of 1-(5-bromothiophen-2-yl)ethan-1-one (10.0 g, 48.8 mmol, 1.00 eq.) and (R)-2-methylpropane-2-sulfinamide (7.68 g, 63.4 mmol, 1.30 eq.) in THF (120 mL) was added titanium (IV) ethoxide (22.3 g, 97.5 mmol, 20.2 mL, 2.00 eq.), the reaction mixture was stirred at 70° C. for 12 hours under a nitrogen atmosphere. The reaction mixture was cooled to 25° C., diluted with water (200 mL) and ethyl acetate (100 mL) to give a suspension, the suspension was filtered and the filtrate was extracted with ethyl acetate (100 mL × 3). The combined organic layers were dried over sodium sulfate, filtered, and concentrated under reduced pressure to give (R, E)-N-(1-(5-bromothiophen-2-yl)ethylidene)-2-methylpropane-2-sulfinamide (13.0 g, crude) as a brown oil. LCMS [M+1]: 308.2.

¹H NMR (400 MHz, CDCl₃) δ = 7.23 (d, J= 4.0 Hz, 1H), 7.04 (d, J= 4.0 Hz, 1H), 2.67 (s, 3H), 1.28 (s, 9H).

Step B: To a solution of (R, E)-N-(I-(5-bromothiophen-2-yl)ethylidene)-2-methylpropane-2-sulfinamide (13.0 g, 42.2 mmol, 1.00 eq.) in THF (150 mL) was added sodium borohydride (4.79 g, 127 mmol, 3.00 eq.) at 0° C. The reaction mixture was stirred at 20° C. for 2 hours under a nitrogen atmosphere. Saturate sodium bicarbonate aqueous solution (20.0 mL) was added to the mixture dropwise and diluted with water (200 mL), the resulting aqueous solution was extracted with ethyl acetate (100 mL X 3), the combined organic layers were dried over sodium sulfate, filtered, and concentrated under vacuum to give a residue. The residue was purified by column chromatography (SiO₂, petroleum ether/ethyl acetate = 30/1 to 2/1) to give (R)-N-((R)-1-(5-bromothiophen-2-yl)ethyl)-2-methylpropane-2-sulfinamide (6.00 g, 17.4 mmol, 41.3% yield, 90.0% purity) as a brown solid. LCMS [M+1]: 309.9.

¹H NMR (400 MHz, CDCl₃) δ = 6.90 (d, J= 3.6 Hz, 1H), 6.80 (d, J= 3.6 Hz, 1H), 4.84 - 4.66 (m, 1H), 3.50 (d, J= 2.8 Hz, 1H), 1.57 (d, J = 6.4 Hz, 3H), 1.23 (s, 9H).

Step C: To a solution of (R)-N-((R)-1-(5-bromothiophen-2-yl)ethyl)-2-methylpropane-2-sulfinamide (2.00 g, 6.45 mmol, 1.00 eq.) and tert-butyl methyl(2-(4,4,5,5-tetramethyl-1,3,2-dioxaborolan-2-yl)benzyl)carbamate (2.69 g, 7.74 mmol, 1.20 eq.) in dioxane (20.0 mL) and water (2.00 mL) was added cesium carbonate (6.30 g, 19.3 mmol, 3.00 eq.) and Pd(PPh₃)₄ (745 mg, 645 µmol, 0.10 eq.) under a nitrogen atmosphere. The reaction mixture was stirred at 110° C. for 2 hours under a nitrogen atmosphere. The reaction mixture was then cooled to 25° C., diluted with water (100 mL), and extracted with ethyl acetate (50.0 mL× 3). The combined organic layers were dried over sodium sulfate, filtered, and concentrated under reduced pressure to give a residue. The residue was purified by column chromatography (SiO₂, petroleum ether/ethyl acetate = 20/1 to 1/1) to give tert-butyl (2-(5-((R)-1-(((R)-tert-butylsulfinyl)amino)ethyl)thiophen-2-yl)benzyl)(methyl)carbamate (2.60 g, 5.19 mmol, 80.6% yield, 90.0% purity) as a yellow oil. LCMS [M+1]: 451.4.

¹H NMR (400 MHz, CDCl₃) δ = 7.40 - 7.32 (m, 2H), 7.31 - 7.27 (m, 1H), 7.26 - 7.22 (m, 1H), 7.01 (s, 1H), 6.83 (s, 1H), 4.95 - 4.79 (m, 1H), 4.67 - 4.44 (m, 2H), 3.56 (d, J = 3.2 Hz, 1H), 2.93 - 2.56 (m, 3H), 1.64 (d, J = 6.4 Hz, 3H), 1.56 - 1.36 (m, 9H), 1.26 (s, 9H).

Step D: To a solution of tert-butyl (2-(5-((R)-1-(((R)-tert-butylsulfinyl)amino)ethyl)thiophen-2-yl)benzyl)(methyl)carbamate (2.60 g, 5.77 mmol, 1.00 eq.) in THF (20.0 mL) and water (4.00 mL) was added iodine (439 mg, 1.73 mmol, 349 µL, 0.30 eq.), the reaction mixture was stirred at 50° C. for 2 hours. The reaction mixture was cooled to 25° C., diluted with saturate sodium bicarbonate (50.0 mL) and extracted with ethyl acetate (20.0 mL × 3). The combined organic layers were dried over sodium sulfate, filtered, and concentrated under reduced pressure to give a residue. The residue was purified by column chromatography (SiO₂, petroleum ether/ethyl acetate = 10/1 to 0/1) to give (R)- tert-butyl (R)-(2-(5-(1-aminoethyl)thiophen-2-yl)benzyl)(methyl)carbamate (1.50 g, 3.68 mmol, 63.8% yield, 85.0% purity) as a yellow oil. LCMS [2M+1]: 693.3.

¹H NMR (400 MHz, CDCl₃) δ = 7.39 - 7.31 (m, 2H), 7.30 - 7.20 (m, 2H), 7.01 (d, J = 2.8 Hz, 1H), 6.81 (d, J= 3.2 Hz, 1H), 4.61 - 4.48 (m, 3H), 4.04 (s, 2H), 2.73 (s, 3H), 1.64 (d, J = 6.4 Hz, 3H), 1.57 - 1.33 (m, 9H).

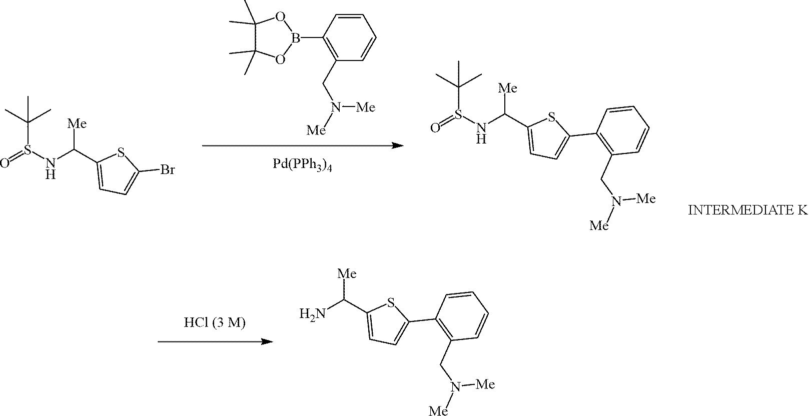

Step A: To a solution of N-(1-(5-bromothiophen-2-yl)ethyl)-2-methylpropane-2-sulfinamide (0.50 g, 1.61 mmol, 1.00 eq.) and N, N-dimethyl-1-(2-(4,4,5,5-tetramethyl-1,3,2-dioxaborolan-2-yl)phenyl)methanamine (505 mg, 1.93 mmol, 1.20 eq.) in dioxane (5.00 mL) and water (0.50 mL) was added cesium carbonate (1.58 g, 4.83 mmol, 3.00 eq.) and Pd(PPh₃)₄ (186 mg, 161 µmol, 0.10 eq.), then degassed and purged with nitrogen 3 times. The reaction mixture was stirred at 110° C. for 2 hours under a nitrogen atmosphere. Upon completion, the reaction mixture was cooled to 25° C., diluted with water (50.0 mL) and extracted with ethyl acetate (20.0 mL × 3). The combined organic layers were dried over anhydrous sodium sulfate, filtered, and concentrated under reduced pressure to give a residue. The residue was purified by column chromatography (SiO₂, petroleum ether/ethyl acetate = 20/1 to 0/1) to give N-(1-(5-(2-((dimethylamino)methyl)phenyl)thiophen-2-yl)ethyl)-2-methylpropane-2-sulfinamide (450 mg, 1.15 mmol, 71.3% yield, 93.0% purity) as a brown oil. LCMS [M+1]: 365.2.

Step B: To a solution of N-(1-(5-(2-((dimethylamino)methyl)phenyl)thiophen-2-yl)ethyl)-2-methylpropane-2-sulfinamide (410 mg, 1.12 mmol, 1.00 eq.) in THF (4.00 mL) was added hydrochloric acid (3.00 M, 375 µL, 1.00 eq.), the reaction mixture was stirred at 20° C. for 2 hours. Upon completion, the reaction mixture was diluted with saturated sodium bicarbonate (50.0 mL) and extracted with ethyl acetate (20.0 mL × 3). The combined organic layers were dried over anhydrous sodium sulfate, filtered, and concentrated under reduced pressure to give a residue. The residue was purified by column chromatography (SiO₂, petroleum ether/ethyl acetate = 10/1 to dichloromethane/methanol = 10/1) to give 1-(5-(2-((dimethylamino)methyl)phenyl)thiophen-2-yl)ethanamine (200 mg, 691 µmol, 61.5% yield, 90.0% purity) as a yellow oil.

¹H NMR (400 MHz, DMSO-d₆) δ = 7.48 - 7.42 (m, 1H), 7.41 - 7.36 (m, 1H), 7.34 -7.28 (m, 2H), 7.13 (d, J = 3.6 Hz, 1H), 6.96 - 6.92 (m, 1H), 4.29 - 4.21 (m, 1H), 3.39 (s, 2H), 2.14 (s, 6H), 1.38 (d, J = 6.4 Hz, 3H).

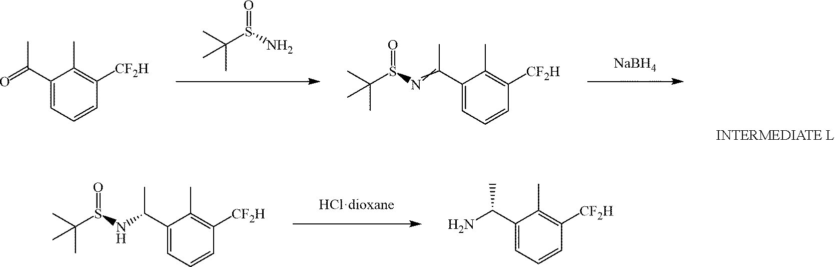

Step A: To a solution of 1-(3-(difluoromethyl)-2-methylphenyl)ethan-1-one (0.37 g, 1.99 mmol, 1.00 eq.) in tetrahydrofuran (10.0 mL) was added titanium(IV) ethoxide (2.27 g, 9.95 mmol, 2.06 mL, 5.00 eq.) and (R)-2-methylpropane-2-sulfinamide (724 mg, 5.97 mmol, 3.00 eq.). The mixture was stirred at 75° C. for 16 hours. The reaction mixture was quenched by addition saturated aqueous sodium bicarbonate 20.0 mL at 25° C. The mixture was filtered, and filtrate was extracted with ethyl acetate 45.0 mL (15.0 mL × 3). The combined organic layers were washed with brine 20.0 mL (20.0 mL × 1), dried over anhydrous sodium sulfate, filtered, and concentrated under reduced pressure to give a residue. The residue was purified by flash silica gel chromatography (0~12% Ethyl acetate/Petroleum ether) to give (R,E)-N-(1-(3-(difluoromethyl)-2-methylphenyl)ethylidene)-2-methylpropane-2-sulfinamide (0.36 g, 1.19 mmol, 59.8% yield, 95.0% purity) as a colorless oil.

¹H NMR (400 MHz, CD₃OD) δ = 7.55 - 7.62 (m, 1H), 7.16 - 7.51 (m, 2H), 6.79 - 7.13 (m, 1H), 2.48 - 2.73 (m, 3H), 2.27 - 2.47 (m, 3H), 1.19 - 1.30 (m, 9H).

Step B: To a solution of (R,E)-N-(1-(3-(difluoromethyl)-2-methylphenyl)ethylidene)-2-methylpropane-2-sulfinamide (340 mg, 1.18 mmol, 1.00 eq.) in tetrahydrofuran (5.00 mL) was added sodium borohydride (89.5 mg, 2.37 mmol, 2.00 eq.). The mixture was stirred at 0° C. for 1 hour. The reaction mixture was quenched by addition water 10.0 mL at 25° C., and then extracted with ethyl acetate 30.0 mL (10.0 mL × 3). The combined organic layers were washed with brine (10.0 mL × 1) dried over anhydrous sodium sulfate, filtered, and concentrated under reduced pressure to give a residue. The residue was purified by flash silica gel chromatography (0-13% Ethyl acetate/Petroleum ether) to give (R)-N-((R)-1-(3-(difluoromethyl)-2-methylphenyl)ethyl)-2-methylpropane-2-sulfinamide (190 mg, 643 µmol, 54.4% yield, 98.0% purity) as a yellow oil. LCMS [M+1] ⁺= 290.1.

Step C: A mixture of (R)-N-((R)-1-(3-(difluoromethyl)-2-methylphenyl)ethyl)-2-methylpropane-2-sulfinamide (140 mg, 484 µmol, 1.00 eq.) in dioxane hydrochloride (4.00 M, 7.00 mL, 57.9 eq) was stirred at 25° C. for 1 h. The reaction mixture was concentrated under reduced pressure to give crude product (R)-1-(3-(difluoromethyl)-2-methylphenyl)ethan-1-amine (110 mg, 475 µmol, 98.2% yield, 80.0% purity) as a white solid, which was used without further purification. LCMS [M+1] ⁺= 186.0.

The following Examples are intended to illustrate further certain embodiments of the invention and are not intended to limit the scope of the invention.

### Example 1-1

(R)((1-(2-methyl-3-(trifluoromethyl)phenyl)ethyl)amino)-6-morpholinophthalazin-1(2H)-one

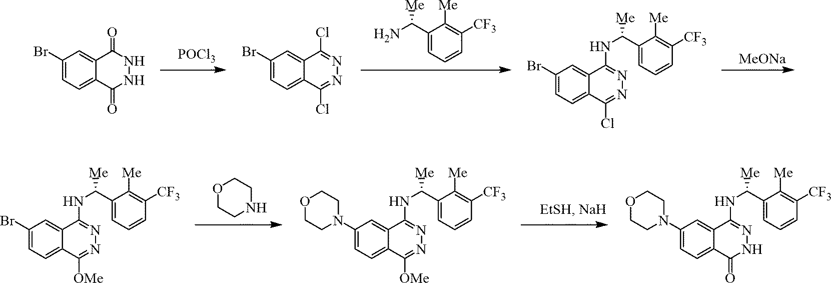

Step A: To a solution of 6-bromo-2,3-dihydrophthalazine-1,4-dione (3.00 g, 12.4 mmol, 1.00 eq.) in phosphorus oxychloride (40.0 mL) was added N, N-diisopropylethylamine (4.02 g, 31.1 mmol, 5.42 mL, 2.50 eq.) dropwise at 25° C. The reaction was then stirred at 120° C. for 12 hours. The mixture was cooled to 25° C. and concentrated in vacuo to remove most of the phosphorus oxychloride and give a residue. The residue was poured into ice water (100 mL), and the resulting aqueous solution was adjusted to pH = 7 with saturated sodium bicarbonate aqueous solution and then extracted with dichloromethane (50.0 mL × 2). The combined organic phases were washed with brine (30.0 mL × 2), dried over anhydrous sodium sulfate, filtered, and concentrated under vacuum to give 6-bromo-1,4-dichlorophthalazine (1.20 g, 4.32 mmol, crude) as a yellow solid without further purification. LCMS [M+3]⁺: 279.0.

Step B: To a mixture of 6-bromo-1,4-dichlorophthalazine (500 mg, 1.80 mmol, 1.00 eq.) and (R)-1-(2-methyl-3-(trifluoromethyl)phenyl)ethan-1-amine (365 mg, 1.80 mmol, 1.00 eq.) in DMSO (10.0 mL) was added potassium fluoride (313 mg, 5.40 mmol, 126 µL, 3.00 eq.), N, N-diisopropylethylamine (465 mg, 3.60 mmol, 627 µL, 2.00 eq.) under a nitrogen atmosphere. The reaction mixture was then stirred at 130° C. for 3 hours. After this time, the reaction was cooled to 25° C., diluted with ethyl acetate (20.0 mL), washed with brine (5.00 mL × 2), dried over anhydrous sodium sulfate, filtered, and concentrated under vacuum to give a residue. The residue was purified by preparative TLC (petroleum ether/ethyl acetate = 3/1) to give (R)-7-bromo-4-chloro-N-(1-(2-methyl-3-(trifluoromethyl)phenyl)ethyl)phthalazin-1-amine (360 mg, 769 µmol, 42.7% yield) as a white solid. LCMS [M+3] ⁺_{:} 446.1.

¹HNMR (400 MHz, CDCl₃) δ = 8.15 - 8.01 (m, 2H), 7.99 - 7.79 (m, 1H), 7.63 (d, J = 8.0 Hz, 1H), 7.56 - 7.50 (m, 1H), 7.23 (s, 1H), 5.91 - 5.77 (m, 1H), 5.45 (br d, J = 6.4 Hz, 1H), 2.55 (s, 3H), 1.65 (d, J = 6.8 Hz, 3H).

Step C: To a mixture of (R)-7-bromo-4-chloro-N-(1-(2-methyl-3-(trifluoromethyl)phenyl)ethyl)phthalazin-1-amine (330 mg, 742 µmol, 1.00 eq.) in methanol (5.00 mL) was added sodium methoxide (200 mg, 3.71 mmol, 5.00 eq.) under nitrogen. The reaction mixture was stirred for 2 hours at 110° C. in a microwave reactor. The reaction mixture was then cooled to 25° C., and concentrated under reduced pressure to give a residue. The residue was purified by column chromatography (SiO₂, petroleum ether/ethyl acetate = 50/1 to 1/1) to give (R)-7-bromo-4-methoxy-N-(1-(2-methyl-3-(trifluoromethyl)phenyl)ethyl)phthalazin-1-amine (281 mg, 638 µmol, 86.0% yield) as a white solid.

Step D: To a mixture of (R)-7-bromo-4-methoxy-N-(1-(2-methyl-3-(trifluoromethyl)phenyl)ethyl)phthalazin-1-amine (150 mg, 341 µmol, 1.00 eq.) and morpholine (89.0 mg, 1.02 mmol, 89.9 µL, 3.00 eq.) in toluene (5.00 mL) was added RuPhos (31.8 mg, 68.1 µmol, 0.20 eq.), cesium carbonate (222 mg, 681 µmol, 2.00 eq.), and RuPhos Pd G3 (28.5 mg, 34.1 µmol, 0.10 eq.) under a nitrogen atmosphere. The reaction mixture was stirred at 90° C. for 6 hours. After this time, the reaction mixture was cooled to 25° C., diluted with water (10.0 mL) and extracted with ethyl acetate (10.0 mL × 3). The combined organic layers were washed with brine (10.0 mL), dried over sodium sulfate, filtered, and concentrated under reduced pressure to give a residue. The residue was purified by column chromatography (SiO₂, petroleum ether/ethyl acetate=10/1 to 0/1) to give (R)-4-methoxy-N-(1-(2-methyl-3-(trifluoromethyl)phenyl)ethyl)-7-morpholinophthalazin-1-amine (140 mg, 31.0 µmol, 92.0% yield) as a yellow solid. LCMS [M+1]: 447.3.

¹HNMR (400 MHz, CDCl₃) δ = 8.03 (d, J = 8.8 Hz, 1H), 7.70 (d, J = 7.6 Hz, 1H), 7.56 (d, J = 7.6 Hz, 1H), 7.39 (dd, J = 2.4, 9.2 Hz, 1H), 7.31 - 7.27 (m, 1H), 6.84 (d, J = 2.4 Hz, 1H), 5.85 - 5.81 (m, 1H), 4.61 (br d, J = 6.4 Hz, 1H), 4.13 (s, 3H), 3.94 - 3.84 (m, 4H), 3.43 -3.30 (m, 4H), 2.53 (s, 3H), 1.66 (d, J = 6.4 Hz, 3H).

Step E: To a suspension of sodium hydride (28.7 mg, 717 µmol, 60% purity, 8.00 eq.) in DMF (2.00 mL) was added ethanethiol (630 mg, 10.1 mmol, 750 µL, 113 eq.) under a nitrogen atmosphere. The reaction mixture was stirred at 25° C. for 15 minutes, then a solution of (R)-4-methoxy-N-(1-(2-methyl-3-(trifluoromethyl)phenyl)ethyl)-7-morpholinophthalazin-1-amine (40.0 mg, 89.6 µmol, 1.00 eq.) in dry DMF (2.00 mL) was added to the reaction. The reaction mixture was stirred at 120° C. for 2 hours. At this time, the reaction mixture was cooled to 25° C., diluted with ethyl acetate (20.0 mL) and washed with brine (10.0 mL × 2), dried over sodium sulfate, filtered, and concentrated under reduced pressure to give a residue. The residue was purified by prep-HPLC (column: Waters Xbridge 150 x 25 mm x 5 µm; mobile phase: [water(10 mM NH₄HCO₃) - ACN]; B%: 42% - 72%, 10 min) to give (R)-4-((1-(2-methyl-3-(trifluoromethyl)phenyl)ethyl)amino)-6-morpholinophthalazin-1(2H)-one (5.33 mg, 12.1 µmol, 13.5% yield) as a white solid. LCMS [M+1]: 433.2.

¹HNMR (400 MHz, CDCl₃) δ = 9.02 (s, 1H), 8.29 (d, J = 8.8 Hz, 1H), 7.64 (d, J = 7.6 Hz, 1H), 7.55 (d, J = 7.6 Hz, 1H), 7.29 (dd, J = 2.4, 9.2 Hz, 1H), 6.85 (d, J = 2.4 Hz, 1H), 5.34 -5.26 (m, 1H), 4.47 (br d, J = 5.2 Hz, 1H), 3.95 - 3.87 (m, 4H), 3.44 - 3.35 (m, 4H), 2.53 (s, 3H), 1.58 (d, J = 6.8 Hz, 3H).

SFC conditions: Column: Chiralcel OD-3 50×4.6 mm I.D., 3 µm, Mobile phase: Phase A for CO₂, and Phase B for MeOH (0.05%DEA); Gradient elution: MeOH (0.05% DEA) in CO₂ from 5% to 40%, Flow rate: 3 mL/min; Detector: PDA, Column Temp: 35C; Back Pressure: 100Bar.

### Example 1-2

(R)((1-(4-(2-((methylamino)methyl)phenyl)thiophen-2-yl)ethyl)amino)-6-morpholinophthalazin-1 (2H)-one

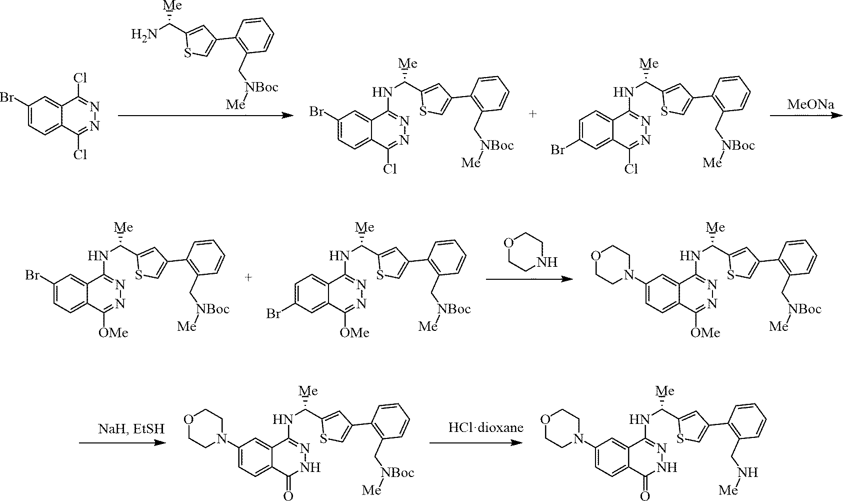

Step A: To a solution of 6-bromo-1,4-dichlorophthalazine (550 mg, 1.98 mmol, 1.00 eq.), tert-butyl (R)-(2-(5-(1-aminoethyl)thiophen-3-yl)benzyl)(methyl)carbamate (686 mg, 1.98 mmol, 1.00 eq.) in dimethylsulfoxide (10.0 mL) was added potassium fluoride (345 mg, 5.94 mmol, 139 µL, 3.00 eq.), and diisopropylethylamine under a nitrogen atmosphere. The reaction mixture was stirred at 130° C. for 2 hour, then cooled to 25° C., diluted with ethyl acetate (40.0 mL), washed with brine (20.0 mL×2), dried over anhydrous sodium sulfate, filtered, and concentrated under reduced pressure to give the residue. The residue was purified by prep-HPLC (column: Waters Xbridge C18 150 X 50 mm X 10 µm; mobile phase: phase A: [water(10 mM NH₄HCO₃), phase B: acetonitrile]; B%: 64% - 94%, 11.5 min) to give two isomers tert-butyl (R)-(2-(5-(1-((7-bromo-4-chlorophthalazin-1-yl)amino)ethyl)thiophen-3-yl)benzyl)(methyl)carbamate and tert-butyl (R)-(2-(5-(1-((6-bromo-4-chlorophthalazin-1-yl)amino)ethyl)thiophen-3-yl)benzyl)(methyl)carbamate (380 mg, 646 µmol, 32.6% yield) as a light yellow solid. LCMS [M+3] ⁺_{:} 588.8.

Step B: To a solution of two isomers tert-butyl (R)-(2-(5-(1-((7-bromo-4-chlorophthalazin-1-yl)amino)ethyl)thiophen-3-yl)benzyl)(methyl)carbamate and tert-butyl (R)-(2-(5-(1-((6-bromo-4-chlorophthalazin-1-yl)amino)ethyl)thiophen-3-yl)benzyl)(methyl)carbamate (360 mg, 612 µmol, 1.0 eq.) in methanol (8.00 mL) was added sodium methoxide (122 mg, 3.06 mmol, 10.0 eq.), the reaction was stirred at 110° C. for 1 hour in a microwave reactor. The reaction was filtered and concentrated under reduced pressure to give a residue. The residue was purified by column chromatography (SiO₂, petroleum ether/ethyl acetate=6/1 to 2/1) to give two isomers tert-butyl (R)-(2-(5-(1-((7-bromo-4-methoxyphthalazin-1-yl)amino)ethyl)thiophen-3-yl)benzyl)(methyl)carbamate and tert-butyl (R)-(2-(5-(1-((6-bromo-4-methoxyphthalazin-1-yl)amino)ethyl)thiophen-3-yl)benzyl)(methyl)carbamate (250 mg, 428 µmol, 70.0% yield) as a yellow solid. LCMS [M+3] ⁺ : 585.2.

Step C: To a solution of two isomers (tert-butyl (R)-(2-(5-(1-((7-bromo-4-methoxyphthalazin-1-yl)amino)ethyl)thiophen-3-yl)benzyl)(methyl)carbamate and tert-butyl (R)-(2-(5-(1-((6-bromo-4-methoxyphthalazin-1-yl)amino)ethyl)thiophen-3-yl)benzyl)(methyl)carbamate (250 mg, 428 µmol, 1.00 eq.), morpholine (54.0 mg, 617 µmol, 54.0 µL, 3.00 eq.) in dioxane (6.00 mL) was added Pd₂(dba)₃ (18.8 mg, 20.6 µmol,0.10 eq.), RuPhos (19.2 mg, 41.1 µmol, 0.20 eq.), cesium carbonate (134 mg, 411 µmol, 2.00 eq.) under a nitrogen atmosphere, and the reaction was stirred at 100° C. for 3 hours under a nitrogen atmosphere. The reaction was cooled to 25° C. and concentrated under reduced pressure to give a residue. The residue was purified by column chromatography (SiO₂, petroleum ether/ethyl acetate = 4/1 to 1/1) to give tert-butyl (R)-(2-(5-(1-((4-methoxy-7-morpholinophthalazin-1-yl)amino)ethyl)thiophen-3-yl)benzyl)(methyl)carbamate (95.0 mg, 161 µmol, 39.1 % yield) as a white solid. LCMS [M+1] ⁺_{:} 590.3.

Step D: Ethanethiol (42.1 mg, 678 µmol, 50.2 µL, 20.0 eq.) was added to a suspension of sodium hydride (13.5 mg, 338 µmol, 60% purity, 10.0 eq.) in DMF (0.40 mL) under an atmosphere of nitrogen. The reaction mixture is stirred for 5 min before a solution of tert-butyl (R)-(2-(5-(1-((4-methoxy-7-morpholinophthalazin-1-yl)amino)ethyl)thiophen-3-yl)benzyl)(methyl)carbamate (20.0 mg, 33.9 µmol, 1.00 eq.) in DMF (0.40 mL) were added. The reaction was then stirred at 100° C. for 2 hours and stirred at 120° C. for another 2 hours. The mixture was cooled, extracted with ethyl acetate (3.00 mL × 3). The organic phases were washed with water (3.00 mL), dried over anhydrous sodium sulfate, filtered, and concentrated under reduced pressure to give tert-butyl (R)-methyl(2-(5-(1-((7-morpholino-4-oxo-3,4-dihydrophthalazin-1-yl)amino)ethyl)thiophen-3-yl)benzyl)carbamate (20.0 mg, crude) as a white solid used directly. LCMS [M+1] ⁺:576.5.

Step E: To a mixture of give tert-butyl (R)-methyl(2-(5-(1-((7-morpholino-4-oxo-3,4-dihydrophthalazin-1-yl)amino)ethyl)thiophen-3-yl)benzyl)carbamate (70.0 mg, 122 µmol, 1.00 eq.) in acetonitrile (1.00 mL) was added HCl•dioxane (0.50 mL) dropwise and then the mixture was stirred at 0° C. for 30 minutes. To the mixture was added methanol (1.00 mL) at 0° C. and adjusted to pH=7 with solid sodium bicarbonate (around 30.0 mg) to give a suspension. The suspension was filtered, and the filtrate was concentrated under reduced pressure to give a residue. The residue was purified by prep-HPLC (column: Welch Xtimate C18 150 X 30 mm × 5 µm; mobile phase: phase A: [water (0.05% ammonia hydroxide v/v), phase B: acetonitrile]; B%: 28% - 58%, 11.5 min) to give (R)-4-((1-(4-(2-((methylamino)methyl)phenyl)thiophen-2-yl)ethyl)amino)-6-morpholinophthalazin-1(2H)-one (33.3 mg, 69.1 µmol, 56.8% yield, 98.7% purity) as white solid. LCMS [M+1] ⁺ : 476.5.

¹H NMR (400 MHz, CD₃OD) δ = 8.21 - 8.14 (m, 1H), 7.48 - 7.38 (m, 3H), 7.35 - 7.25 (m, 3H), 7.15 (d, J=1.6 Hz, 1H), 7.13 - 7.10 (m, 1H), 5.47 (q, J = 6.8 Hz, 1H), 3.93 - 3.84 (m, 4H), 3.72 (s, 2H), 3.50 - 3.42 (m, 4H), 2.22 (s, 3H), 1.76 (d, J = 6.8 Hz, 3H).

### Example 1-3

(R)methyl-4-((1-(4-(2-((methylamino)methyl)phenyl)thiophenyl)ethyl)amino)-6-morpholinophthalazin-1 (2H)-one

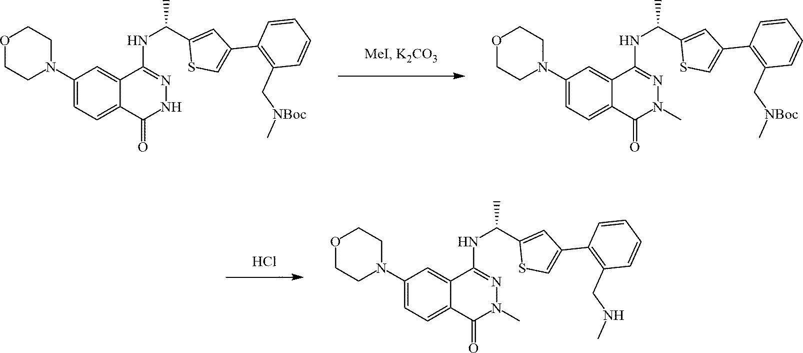

Step A: To a suspension of tert-butyl (R)-methyl(2-(5-(1-((7-morpholino-4-oxo-3,4-dihydrophthalazin-1-yl)amino)ethyl)thiophen-3-yl)benzyl)carbamate (25.0 mg, 43.4 µmol, 1.00 eq.) in dry DMF (0.30 mL) was added potassium carbonate (30.0 mg, 217 µmol,5.00 eq.). A solution of methyl iodide (49.3 mg, 347 µmol, 21.6 µL, 8.00 eq.) in DMF (0.40 mL) (about 140 mg/mL) was then added dropwise to the reaction mixture. The reaction was stirred at 100° C. for 12 hours and stirred at 120° C. for another 5 hours. The reaction mixture was then cooled to 25° C., quenched by a saturated solution of ammonium chloride (1.50 mL). Then extracted with ethyl acetate (3.00 mL×3), The organic layers were washed with brine (3.00 mL), dried over anhydrous sodium sulfate, and concentrated under reduced pressure to give a solid residue. The residue was purified by prep-HPLC (column: Waters Xbridge 150 × 25 mm × 5 µm; mobile phase: phase A: [water(10 mM NH₄HCO₃), phase B: acetonitrile]; B%: 50% - 80%, 9 min) to give tert-butyl (R)-methyl(2-(5-(1-((3-methyl-7-morpholino-4-oxo-3,4-dihydrophthalazin-1-yl)amino)ethyl)thiophen-3-yl)benzyl)carbamate (5.00 mg, 8.48 µmol, 19.5% yield) as a yellow oil used directly. LCMS [M+1] ⁺ : 590.2.

¹H NMR (400 MHz, DMSO-d₆) δ = 8.11 (d, J=9.6 Hz, 1H), 7.54 - 7.46 (m, 2H), 7.44 -7.38 (m, 1H), 7.35 (br d, J =4.0 Hz, 3H), 7.20 (br d, J =6.8 Hz, 2H), 5.52 - 5.37 (m, 1H), 4.50 (s, 2H), 3.89 - 3.78 (m, 4H), 3.57 (s, 3H), 3.46 - 3.43 (m, 4H), 2.74 (s, 3H), 1.75 (d, J =6.8 Hz, 3H), 1.50 - 1.29 (m, 9H).

Step B: To a mixture of tert-butyl (R)-methyl(2-(5-(1-((3-methyl-7-morpholino-4-oxo-3,4-dihydrophthalazin-1-yl)amino)ethyl)thiophen-3-yl)benzyl)carbamate (5.00 mg, 8.48 µmol, 1.00 eq.) in acetonitrile (0.60 mL) was added HCl·dioxane (4 M, 0.10 mL) dropwise and then the mixture was stirred at -10° C. for 30 minutes. To the mixture was added methanol (0.50 mL) at -10° C., adjusted to pH = 7 with solid sodium bicarbonate (around 5 mg) to give a suspension, the suspension was filtered, the filtrate was concentrated to give a residue. The residue was purified by prep-HPLC (column: Waters Xbridge 150 × 25 mm × 5 µm; mobile phase: phase A: [water (0.05% ammonia hydroxide v/v), phase B: acetonitrile]; B%: 35% - 65%, 10 min) to give ((R)-2-methyl-4-((1-(4-(2-((methylamino)methyl)phenyl)thiophen-2-yl)ethyl)amino)-6-morpholinophthalazin-1(2H)-one (1.00 mg, 1.99 µmol, 23.5% yield, 97.6% purity) as a white solid. LCMS [M+1]⁺ : 490.2.

¹H NMR (400 MHz, CD₃OD) δ = 8.17 (d, J= 8.8 Hz, 1H), 7.54 - 7.49 (m, 1H), 7.48 -7.43 (m, 3H), 7.41 (d, J= 2.4 Hz, 2H), 7.21 (d, J = 1.6 Hz, 1H), 7.12 (s, 1H), 5.54 - 5.44 (m, 1H), 4.20 (br s, 2H), 3.93 - 3.81 (m, 4H), 3.65 (s, 3H), 3.48 - 3.40 (m, 4H), 2.50 (d, J= 2.4 Hz, 3H), 1.77 (d, J=7.2 Hz, 3H).

Using the intermediates and following the teachings of General Reaction Schemes I -V and EXAMPLES 1-1 - 1-3, EXAMPLES 1-4, 1-5 and 1-6 were prepared as shown in Table 1.

TABLE 1

Examples 1-4, 1-5, and 1-6

| Example No. | Structure | Spectral Data |
|---|---|---|
| 1-4 | 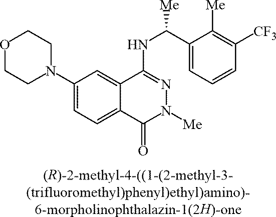 | ¹H NMR (400 MHz, CDCl₃) δ = 8.30 (d, J = 8.8 Hz, 1H), 7.63 (d, J = 8.0 Hz, 1H), 7.54 (d, J = 7.2 Hz, 1H), 7.30 - 7.27 (m, 1H), 7.25 - 7.21 (m, 1H), 6.81 (d, J = 2.4 Hz, 1H), 5.36 - 5.25 (m, 1H), 4.48 (br d, J = 4.8 Hz, 1H), 3.95 - 3.90 (m, 4H), 3.54 (s, 3H), 3.41 - 3.36 (m, 4H), 2.60 (s, 3H), 1.57 (br s, 3H). LCMS [M+1] ⁺_{:} 447.3 |
| 1-5 | 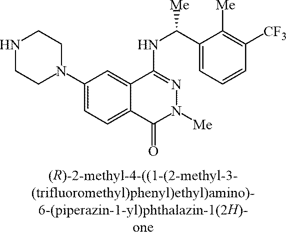 | ¹H NMR (400 MHz, CD₃OD) δ = 8.16 (d, J = 8.8 Hz, 1H), 7.72 (d, J = 7.6 Hz, 1H), 7.61 (d, J = 2.0 Hz, 1H), 7.51 - 7.46 (m, 2H), 7.25 (t, J = 7.6 Hz, 1H), 5.32 (q, J= 6.8 Hz, 1H), 3.81 - 3.75 (m, 4H), 3.47 (s, 3H), 3.46 -3.41 (m, 4H), 2.62 (s, 3H), 1.58 (d, J = 7.2 Hz, 3H). LCMS [M+1] ⁺_{:} |
| | | 446.3. |
| 1-6 | 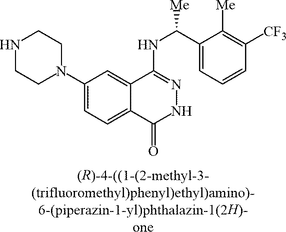 | ¹H NMR (400 MHz, CD₃OD) δ = 8.51 (s, 1H), 8.14 (d, J = 9.2 Hz, 1H), 7.70 (d, J = 8.0 Hz, 1H), 7.56 (d, J= 2.0 Hz, 1H), 7.49 (d, J= 7.6 Hz, 1H), 7.46 - 7.41 (m, 1H), 7.25 (t, J = 8.0 Hz, 1H), 5.37 - 5.28 (m, 1H), 3.77 - 3.67 (m, 4H), 3.39 -3.33 (m, 4H), 2.57 (s, 3H), 1.56 (d, J = 6.8 Hz, 3H). LCMS [M+1] ⁺_{:} 432.2 |

### Example A

This Example illustrates that exemplary compounds of the present invention bind to SOS1 and prevent a labeled tracer ligand from occupying the SOS1 binding site.

The ability of a compound of Formula (I) to bind to SOS1 was measured using a HTRF displacement assay. A recombinant human SOS1 polypeptide (corresponding to amino acids 560-1049, expressed in E. Coli with N-terminal His-TEV-AviTag-SOS1 (MW=59.4 kDa) and lanthanide labeled streptavidin (CisBio) was incubated with an exemplary compound of Formula (I) (in a DMSO stock solution) in buffer (25 mM HEPES pH 7.5, 25 mM NaCl, 1 mM DTT, 0.01% Brij 35, 0.02% BSA, 0.1% DMSO) containing custom-made Cy5 labelled tracer. After a 1-hour incubation at room temperature, the HTRF signal was measured using Clairostar plate reader (BMG Labtech) according to the manufacturer’s instructions. Excitation filter EX-TR was used, and emission 1 was detected at 650-610 nm and emission 2 detected at 620-610 nm. The HTRF ratio was calculated using the formula: [emission ⅟emission 2]*10000.

Background signals were calculated from well with a 10 µM inhibitor, known to inhibit 100% at that concentration. The background subtracted signals were converted to % binding relative to DMSO controls. Data were analyzed using XLFIT software (IDBS) using a Morrison equation for competitive binding and Ki’s were generated for compound of Formula (I).

The results are shown in Table 2.

TABLE 2

| Example No. | Ki |
|---|---|
| 1-1 | 60 |
| 1-2 | 10 |
| 1-3 | 14 |
| 1-4 | 3000 |
| 1-5 | 32 |
| 1-6 | 11 |

As shown in Table 2, exemplary compounds of the present invention potently inhibited the binding of a SOS1 labeled tracer to SOS1 protein.

### Example B

This Example illustrates that exemplary compounds of the present invention prevent KRas-mediated GTP nucleotide exchange mediated by SOS1 to inhibit KRas activity thereby inhibiting the generation of the downstream effector pERK.

MKN1 cells (15,000/w) or H358 (30,000/w) were seeded in a black clear flat bottom 96-well cell culture plate (Corning, #3904) and incubated at 37° C. overnight. Assay day 1, cells were dosed with compounds of Formula (I) with a 10 µm starting concentration and serially diluted 3x for a total of 9 concentrations. The cells were incubated for 1 hour with the compounds solubilized in DMSO at 37° C. Cells were immediately fixed by adding 50 µL of 4% formaldehyde to all wells in a fume hood and the plates were incubated for 20 minutes at room temperature. The formaldehyde was discarded from the plates and 150 µL of ice-cold methanol was added to permeabilize the cells for 10 minutes at -20° C. The methanol was discarded from each of the plates and any liquid remaining in the plate by tapping the plate against paper towels. Cells were then blocked with 150 µL of Odyssey blocking buffer (LI-COR Biosciences #927-50010) using 0.05% Tween for 1 hour at room temperature on a shaker. The blocking buffer was discarded and 50 µL of primary antibodies pERK (cell signaling Technology #9101L; Rabbit, 1:500) and GapDH (Millipore #MAB34; Mouse, 1:5000) diluted in Odyssey blocking buffer was added. The plates were incubated overnight at 4° C. on a shaker.

On Assay day 2, the primary antibody solution was removed. Each plate was washed 3x times with 150 µL of 1x PBST (PBS + 0.1 % Tween 20) and incubated with 50 µL of secondary antibodies: Anti-Rabbit (LI-COR Biosciences #926-32211) and Anti-Mouse (LI-COR Biosciences #68070) at 1:800 dilution in Odyssey blocking buffer with Tween at room temperature on a shaker for 2 hours (protected from light). The secondary antibody solution as removed and each plate was washed with PBST 3x times. Any liquid remaining was discarded and the plate was imaged using the Licor Odyssey machine according to the manufacturer’s instruction, using a set focus length at 3 mm and both 800 nm and 700 nm filters. The GAPDH normalized scan values for each well were divided by the average of vehicle wells to get the % of pERK inhibition. The IC₅₀ values were then calculated with the Graph pad Prism software.

The results are shown in Table 3. Key: N.D. = not determined.

TABLE 3

| Example No. | IC₅₀ |
|---|---|
| 1-1 | 1316 |
| 1-2 | 202 |
| 1-3 | 265 |
| 1-4 | N.D. |
| 1-5 | 881 |
| 1-6 | 114 |

The results in Table 3 illustrate that the compounds of the present invention are capable of potently inhibiting KRas-mediate activation and formation of pERK thereby blocking intracellular KRas-mediated signaling.

### Example C

This Example illustrates that exemplary compounds of the present invention prevent KRas-mediated GTP nucleotide exchange mediated by SOS1, in a SOS1 N233Y mutant cell line, to inhibit KRas activity thereby inhibiting the generation of the downstream effector pErk.

SOS1 N233Y mutant cells LXF289, RL952 and OCIAML-5 (15,000/w) are seeded in a black clear flat bottom 96-well cell culture plate (Corning, #3904) and incubated at 37° C. overnight. Assay day 1, cells are dosed with compounds of Formula (I) with a 10 µm starting concentration and serially diluted 3x for a total of 9 concentrations. The cells are incubated for 1 hour with the compounds solubilized in DMSO at 37° C. Cells are immediately fixed by adding 50 µL of 4% formaldehyde to all wells in a fume hood and the plates are incubated for 20 minutes at room temperature. The formaldehyde is discarded from the plates and 150 µL of ice-cold methanol is added to permeabilize the cells for 10 minutes at -20° C. The methanol is discarded from each of the plates and any liquid remaining in the plate by tapping the plate against paper towels. Cells are then blocked with 150 µL of Odyssey blocking buffer (LI-COR Biosciences #927-50010) using 0.05% Tween for 1 hour at room temperature on a shaker. The blocking buffer is discarded and 50 µL of primary antibodies pERK (cell signaling Technology #9101L; Rabbit, 1:500) and GapDH (Millipore #MAB34; Mouse, 1:5000) diluted in Odyssey blocking buffer is added. The plates are incubated overnight at 4° C. on a shaker.

On Assay day 2, the primary antibody solution is removed. Each plate is washed 3x times with 150 µL of 1x PBST (PBS + 0.1 % Tween 20) and incubated with 50 µL of secondary antibodies: Anti-Rabbit (LI-COR Biosciences #926-32211) and Anti-Mouse (LI-COR Biosciences #68070) at 1:800 dilution in Odyssey blocking buffer with Tween at room temperature on a shaker for 2 hours (protected from light). The secondary antibody solution is removed and each plate is washed with PBST 3x times. Any liquid remaining is discarded and the plate is imaged using the Licor Odyssey machine according to the manufacturer’s instruction, using a set focus length at 3 mm and both 800 nm and 700 nm filters. The GAPDH normalized scan values for each well is divided by the average of vehicle wells to get the % of pERK inhibition. The IC₅₀ values were then calculated with the Graph pad Prism software.

### Example D

This Example illustrates that exemplary compounds of the present invention prevent increased KRas-mediated GTP nucleotide exchange mediated by SOS1 in NF-1 mutant cell lines to inhibit KRas activity thereby inhibiting the generation of the downstream effector pERK.

A cell line harboring activating mutation in NF-1 gene, NCI-H1435 (ATCC CRL-5870) and a cell line harboring activating mutation in NF-2 gene, NCI-H2052 (ATCC CRL-5915) were employed in these studies. NCI-H1435 and NCI-2052 cells (15,000/w) were seeded in a black clear flat bottom 96-well cell culture plate (Corning, #3904) and incubated at 37° C. overnight. Assay day 1, cells were dosed with compounds of Formula (I) with a 10 µm starting concentration and serially diluted 3x for a total of 9 concentrations. The cells were incubated for 1 hour with the compounds solubilized in DMSO at 37° C. Cells were immediately fixed by adding 50 µL of 4% formaldehyde to all wells in a fume hood and the plates were incubated for 20 minutes at room temperature. The formaldehyde was discarded from the plates and 150 µL of ice-cold methanol was added to permeabilize the cells for 10 minutes at -20° C. The methanol was discarded from each of the plates and any liquid remaining in the plate by tapping the plate against paper towels. Cells were then blocked with 150 µL of Odyssey blocking buffer (LI-COR Biosciences #927-50010) using 0.05% Tween for 1 hour at room temperature on a shaker. The blocking buffer was discarded and 50 µL of primary antibodies pERK (cell signaling Technology #9101L; Rabbit, 1:500) and GapDH (Millipore #MAB34; Mouse, 1:5000) diluted in Odyssey blocking buffer was added. The plates were incubated overnight at 4° C. on a shaker.

On Assay day 2, the primary antibody solution was removed. Each plate was washed 3x times with 150 µL of 1x PBST (PBS + 0.1 % Tween 20) and incubated with 50 µL of secondary antibodies: Anti-Rabbit (LI-COR Biosciences #926-32211) and Anti-Mouse (LI-COR Biosciences #68070) at 1:800 dilution in Odyssey blocking buffer with Tween at room temperature on a shaker for 2 hours (protected from light). The secondary antibody solution as removed and each plate was washed with PBST 3x times. Any liquid remaining was discarded and the plate was imaged using the Licor Odyssey machine according to the manufacturer’s instruction, using a set focus length at 3 mm and both 800 nm and 700 nm filters. The GAPDH normalized scan values for each well were divided by the average of vehicle wells to get the % of pERK inhibition. The IC₅₀ values were then calculated with the Graph pad Prism software.

The results are shown in Table 4.

TABLE 4

| Cell Line | Example No. | IC₅₀ |
|---|---|---|
| NCI-H1435 | 1-1 | 1091 |
| | 1-2 | 311 |
| | 1-3 | 522 |
| NCI-H2052 | 1-1 | >10,000 |
| | 1-2 | 752 |
| | 1-3 | 997 |

The results in Table 4 illustrate that the compounds of the present invention are capable of potently inhibiting KRas-mediated activation and formation of pERK in cells harboring NF-1 or NF-2 activating mutations thereby blocking intracellular KRas-mediated signaling driven by NF-1 driven increased SOS1 activity.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

We claim:

1. A compound of Formula (I): 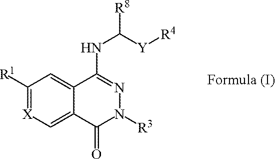
or a pharmaceutically acceptable salt thereof, wherein:
R¹ is hydrogen, hydroxyl, C1 - C6 alkyl, alkoxy, —N(R⁶)₂, —NR⁶C(O)R⁶, —C(O)N(R⁶)₂, SO₂alkyl, -SO₂NR⁶alkyl, cycloalkyl, -Q-heterocyclyl, aryl, or heteroaryl, wherein the cycloalkyl, the heterocyclyl, the aryl, or the heteroaryl are each optionally substituted with one or more R²;
each Q is independently a bond, O or NR⁶;
X is N or CR⁷; with the proviso that when X is N, R¹ is not hydroxyl;
each R² is independently hydroxy, halogen, cyano, hydroxyalkyl, haloalkyl, alkoxy, —N(R⁶)₂, -SO₂alkyl, —NR⁶C(O)C1 — C3 alkyl, -C(O)cycloalkyl, -C(O)heretocyclyl or aryl, wherein the cycloalkyl, the heterocyclyl or the aryl are each optionally substituted with one or more R⁹;
R³ is hydrogen, C1 - C3 alkyl, C1 - C3 haloalkyl, or cycloalkyl;
Y is a bond or heteroarylene;
R⁴ is aryl or heteroaryl, each optionally substituted with one or more R⁵;
each R⁵ is independently hydroxy, halogen, cyano, hydroxyalkyl, alkoxy, C1 - C4 alkyl, haloalkyl, —N(R⁶)₂, —L—N(R⁶)₂ or -SO₂alkyl;
L is C1 - C3 alkylene;
each R⁶ is independently hydrogen, C1 - C3 alkyl, haloalkyl or cycloalkyl;
R⁷ is hydrogen, cyano or alkoxy;
R⁸ is C1 -C2 alkyl or halo-C1 - C2 alkyl; and
each R⁹ is independently C1 - C3 alkyl or haloalkyl.

2. The compound according to claim 1, wherein X is N, with the proviso that when X is N, R¹ is not hydroxyl.

3. The compound according to claim 2, wherein R¹ is alkoxy or -Q-heterocyclyl, wherein the heterocyclyl is optionally substituted with one or more R².

4. The compound according to claim 3, wherein R¹ is -Q-heterocyclyl, and wherein Q is a bond and the heterocyclyl is morpholinyl, piperazinyl, or piperazinone.

5. The compound according to claim 1, wherein X is CR⁷.

6. The compound according to claim 5, wherein R⁷ is hydrogen.

7. The compound according to claim 6, wherein R¹ is hydrogen.

8. The compound according to claim 6, wherein R¹ is hydroxyl.

9. The compound according to claim 6, wherein R¹ is —N(R⁶)₂.

10. The compound according to claim 6, wherein R¹ is —NR⁶C(O)R⁶.

11. The compound according to claim 6, wherein R¹ is —C(O)N(R⁶)₂.

12. The compound according to claim 6, wherein R¹ is cycloalkyl optionally substituted with one or more R².

13. The compound according to claim 12, wherein the cycloalkyl is cyclobutyl, cyclopentyl or cyclohexyl, each optionally substituted with one or more R²_{.}

14. The compound according to claim 13, wherein the cyclobutyl, cyclopentyl or the cyclohexyl are substituted with one R², wherein R² is C1 - C3 alkyl, alkoxy, halogen, hydroxyl or —N(R⁶)₂.

15. The compound according to claim 6, wherein R¹ is -Q-heterocyclyl optionally substituted with one or more R².

16. The compound according to claim 15, wherein Q is a bond and the heterocyclyl is morpholinyl, piperdinyl, piperazinyl, N-methylpiperazinyl, piperazin-2-one, 1-methyl-piperazin-2-one, or 4-methylthiomorpholine 1,1-dioxide.

17. The compound according to claim 16, wherein Q is a bond and the heterocyclyl is pyrrolidinyl or tetrahydropyranyl, each optionally substituted with one or more R².

18. The compound according to claim 17, wherein the pyrrolidinyl or the tetrahydropyranyl are substituted with one R², wherein R² is C1 - C3 alkyl, alkoxy, hydroxyl or —N(R⁶)₂.

19. The compound according to claim 16, wherein Q is a bond and the heterocyclyl is piperazinyl optionally substituted with one or more R².

20. The compound according to claim 19, wherein the piperazinyl is substituted with one R², wherein R² is -C(O)cycloalkyl or -C(O)heterocyclyl, wherein the cycloalkyl or heterocyclyl portion of the -C(O)cycloalkyl or -C(O)heterocyclyl are each optionally substituted with one or more R⁹.

21. The compound according to claim 20, wherein R² is -C(O)cycloalkyl, wherein the cycloalkyl is cyclopropyl substituted with one R⁹, wherein R⁹ is C1 - C3 alkyl.

22. The compound according to claim 20, wherein R² is -C(O)cycloalkyl, wherein the cycloalkyl is cyclopropyl substituted with one R⁹, wherein R⁹ is haloalkyl.

23. The compound according to claim 20, wherein R² is -C(O)heterocyclyl, wherein the heterocyclyl is oxetanyl or tetrahydropyranyl.

24. The compound according to claim 15, wherein Q is a bond and the heterocyclyl is a bicyclic heterocyclyl.

25. The compound according to claim 24, wherein the bicyclic heterocylyl is diazabicyclo[3.2.0]heptan-2-yl, (1R,5R)-2,6-diazabicyclo[3.2.0]heptan-2-yl, diazabicyclo[3.2.0]heptan-6-yl, (1R,5R)-2,6-diazabicyclo[3.2.0]heptan-6-yl or (R)-2-methylhexahydropyrrolo[1,2-a]pyrazin-6(2H)-one.

26. The compound accoding to claim 15, wherein Q is O and the heterocyclyl is azetidinyl, tetrahydrofuranyl, pyrrolidinyl, or piperdinyl.

27. The compound according to claim 6, wherein R¹ is aryl optionally substituted with one or more R².

28. The compound according to claim 27, wherein the aryl is phenyl optionally substituted with one or more R².

29. The compound according to claim 28, wherein the phenyl is substituted with one R², wherein R² is C1 - C3 alkyl, alkoxy, hydroxyl or —N(R⁶)₂.

30. The compound according to claim 6, wherein R¹ is heteroaryl optionally substituted with one or more R².

31. The compound according to claim 30, wherein the heteroaryl is pyrazolyl optionally substituted with one or more R².

32. The compound according to claim 31, wherein the pyrazolyl is substituted with one R², wherein R² is C1 - C3 alkyl, alkoxy, hydroxyl or —N(R⁶)₂.

33. The compound according to claim 5, wherein R⁷ is cyano or alkoxy.

34. The compound according to claim 33, wherein R⁷ is alkoxy, and the alkoxy is methoxy.

35. The compound according to claim 2, wherein Y is heteroarylene.

36. The compound according to claim 35, wherein the heteroarylene is thiophenylene.

37. The compound according to claim 2, wherein Y is a bond.

38. The compound according to claim 35, wherein R⁴ is aryl or heteroaryl, each optionally substituted with one or more R⁵.

39. The compound according to claim 38, wherein R⁴ is aryl optionally substituted with one or more R⁵.

40. The compound according to claim 39, wherein the aryl is phenyl optionally substituted with one or more R⁵.

41. The compound according to claim 40, wherein the phenyl is substituted with one R⁵, wherein R⁵ is C1 - C4 alkyl, haloalkyl, —N(R⁶)₂, -SO₂alkyl, or —L—N(R⁶)₂.

42. The compound according to claim 41, wherein R⁵ is —L—N(R⁶)₂, wherein L is methylene and one R⁶ is hydrogen and the second R⁶ is C1 - C3 alkyl.

43. The compound according to claim 42, wherein the second R⁶ C1 - C3 alkyl is methyl.

44. The compound according to claim 41, wherein R⁵ is —L—N(R⁶)₂, wherein L is methylene and each R⁶ is C1 - C3 alkyl.

45. The compound according to claim 44, wherein each of the R⁶ C1 - C3 alkyl groups is methyl.

46. The compound according to claim 40, wherein the phenyl is substituted with two R⁵, wherein one R⁵ is C1 - C4 alkyl and the second R⁵ is haloalkyl.

47. The compound according to claim 46, wherein C1 - C4 alkyl is methyl and the haloalkyl is trifluoromethyl.

48. The compound according to claim 40, wherein the phenyl is substituted with two R⁵, wherein one R⁵ is C1 - C4 alkyl and the second R⁵ is —L—N(R⁶)₂.

49. The compound according to claim 48, wherein the one R⁵ C1 — C4 alkyl is methyl, and wherein L is a methylene and each R⁶ of the second R⁵ is C1 - C3 alkyl.

50. The compound according to claim 2, wherein R³ is C1 - C3 alkyl.

51. The compound according to claim 50, wherein the C1 - C3 alkyl is methyl, ethyl or isopropyl.

52. The compound according to claim 2, wherein R³ is hydrogen.

53. The compound according to claim 2, wherein R⁸ is haloC1 - C2 alkyl.

54. The compound according to claim 53, wherein the haloC1 - C2 alkyl is fluoromethyl, difluoromethyl or trifluoromethyl.

55. The compound of claim 1, wherein the compound is: 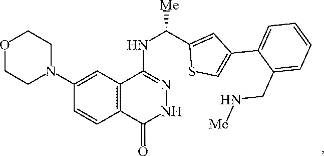 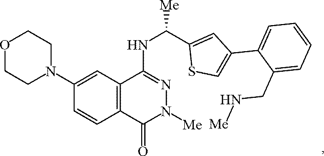 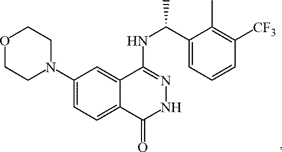 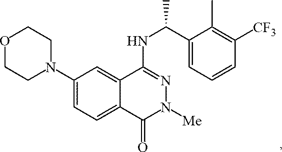 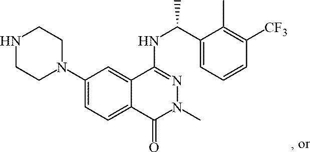 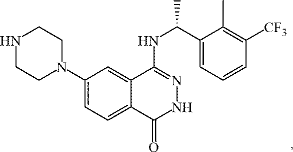
and pharmaceutically acceptable salts thereof.

56. A pharmaceutical composition, comprising a therapeutically effective amount of a compound of Formula (I) according to claim 1 or a pharmaceutically acceptable salt or solvate thereof, and a pharmaceutically acceptable excipient.

57. A method for inhibiting SOS1 activity in a cell, comprising contacting the cell in which inhibition of SOS1 activity is desired with an effective amount of a compound of Formula (I) according to claim 1 or a pharmaceutically acceptable salt or solvate thereof.

58. The method according to claim 57, wherein the cell harbors an activating mutation in a RAS family-member gene.

59. The method according to claim 57, wherein the cell harbors an activating mutation in SOS1 gene.

60. The method according to claim 57, wherein the cell harbors an activating mutation in NF-1 or NF-2 gene.

61. A method for treating cancer comprising administering to a patient having cancer a therapeutically effective amount of a compound of Formula (I) according to claim 1 or a pharmaceutically acceptable salt or solvate thereof, or a pharmaceutically acceptable salt or solvate thereof, alone or combined with a pharmaceutically acceptable carrier, excipient or diluents.

62. The method according to claim 61, wherein the therapeutically effective amount of the compound is between about 0.01 to 300 mg/kg per day.

63. The method according to claim 62, wherein the therapeutically effective amount of the compound is between about 0.1 to 100 mg/kg per day.

64. The method according to claim 61, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi’s sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm’s tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing’s sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial wcarcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin’s disease, non-Hodgkin’s lymphoma (malignant lymphoma); Skin: malignant melanoma, basal cell carcinoma, squamous cell carcinoma, Kaposi’s sarcoma, moles dysplastic nevi, lipoma, angioma, dermatofibroma, keloids, psoriasis; and Adrenal glands: neuroblastoma.

65. The method according to claim 61, wherein the cancer is a Ras family-associated cancer.

66. The method according to claim 65, wherein the Ras family-associated cancer is a KRas, HRas or NRas G12C-associated cancer, a KRas, HRas or NRas G12D-associated cancer, a KRas, HRas or NRas G12S-associated cancer, a KRas, HRas or NRas G12A-associated cancer, a KRas, HRas or NRas G13D-associated cancer, a KRas, HRas or NRas G13C-associated cancer, a KRas, HRas or NRas Q61X-associated cancer, a KRas, HRas or NRas A146T-associated cancer, a KRas, HRas or NRas A146V-associated cancer or a KRas, HRas or NRas A146P-associated cancer.

67. The method according to claim 66, wherein the Ras family-associated cancer is a KRas G12C-associated cancer.

68. The method according to claim 67, wherein the Ras family-associated cancer is non-small cell lung cancer or pancreatic cancer.

69. The method according to claim 61, wherein the cancer is a SOS1-associated cancer.

70. The method according to claim 69, wherein the SOS1-associated cancer is a SOS1 N233S-associated cancer or a SOS1 N233Y-associated cancer.

71. The method according to claim 69, wherein the SOS1-associated cancer is lung adenocarcinoma, embryonal rhabdomyosarcoma, Sertoli cell testis tumor or granular cell tumors of the skin.

72. The method according to claim 61, wherein the cancer is a NF-⅟NF-2-associated cancer.
